(19) United States
(12) Patent Application Publication
(10) Pub. No.: US 2007/0134273 A1
(43) Pub. Date: Jun. 14, 2007

(54) Composition and method for the treatment of carcinoma

(76) Inventors: Francois Romagne, La Ciotat (FR); Jerome Tiollier, Marseille (FR)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 10/588,420

(22) PCT Filed: Feb. 8, 2005
(86) PCT No.: PCT/IB05/00509

§ 371 Date: Aug. 4, 2006

Related U.S. Application Data
(60) Provisional application No. 60/542,858, filed on Feb. 10, 2004.

(51) Int. Cl. A61K 48/00; A61K 38/16; A61K 39/04; A61K 31/7072; A61K 31/7076; A61K 31/663

(52) U.S. Cl. 424/248.1; 514/45; 514/7; 514/54; 514/102

(57) ABSTRACT

The present invention relates to compositions and methods useful for treating a carcinoma or viral infection in mammals, including humans. The methods and compositions typically comprise use of an immunogenic or immunomodulatory compound, and a gamma delta T cell activator, such that the composition is effective for treating a carcinoma or viral infection. In a preferred aspect of the invention, the methods comprise use of a gamma delta T cell activator and a Mycobacterium antigen, which for example is an attenuated strain of Mycobacterium bovis (Bacillus Calmette-Guerin (BCG)).

### FIELD OF THE INVENTION

The present invention relates to compositions and methods useful for treating a carcinoma or viral infection in mammals, including humans. The methods and compositions typically comprise use of an immunogenic or immunomodulatory compound, and a γδT cell activator, such that the composition is effective for treating a carcinoma or viral infection. In a preferred aspect of the invention, the methods comprise use of a γδT cell activator and a Mycobacterium antigen, for example is an attenuated strain of Mycobacterium bovis (Bacillus Calmette-Guérin (BCG)).

### BACKGROUND OF THE INVENTION

Carcinomas account for about 85% of all cancers. A significant portion of these carcinomas are carcinoma in situ, or superficial cancers, such as superficial bladder cancer and diseases caused by human papilloma virus (HPV) infection.

Bladder Cancer

Carcinoma of the bladder accounts for about 2% of all solid tumors in the United States with more than 50,000 new cases being diagnosed each year. The peak prevalence of bladder cancer is in individuals 60-70 years old and several etiologic factors have been implicated including smoking and exposure to industrial chemicals. Bladder cancer is the fifth most common neoplasm and the twelfth leading cause of cancer death.

Pathologically, carcinoma of the bladder is categorized by grade (usually I-IV) and by depth of malignancy (either superficial, invasive, or metastatic bladder cancer). Superficial bladder cancer, which is confined to the bladder epithelium, usually presents as papillary tumors (stages ta or T1) or carcinoma-in-situ (CIS). Diagnosis of bladder cancer is by cytoscopy and biopsy. At the time of diagnosis, about 70% of patients have only superficial disease, 25% have locally invasive disease, and 5% already have distant metastasis.

Superficial bladder cancer is treated with transurethal resection and/or fulguration. Cytoscopy is usually reserved for those tumors which cannot be resected transurethrally. After transurethal resection, 50% of patients remain disease free; however the other half will experience multiple recurrences with about 10% developing invasive or metastatic disease within 3-4 years. Superficial recurrences are treated with transurethal resection, often followed by intravesical chemotherapy to prevent or delay any additional recurrence. Patients who are considered at high risk for recurrence after the initial transurethal resection or those with concurrent CIS are frequently given intravesical adjunct therapy as prophylaxis against recurrence.

High risk patients are candidates for intravesical therapy with bacillus Calmette-Guerin (BCG), mitomycin, doxorubicin or thiotepa. These agents are typically instilled into the bladder through a urethral catheter for two hours weekly for six to eight weeks. Occasionally, long-term maintenance treatment regimens are employed.

Clinical studies may have various endpoints such as tumor recurrence, tumor progression or patient survival. In clinical trials comparing transurethral resection plus and an intravesical agent versus transurethral resection alone, a significant reduction in tumor recurrences was noted in 4 of 5 BCG studies, 2 of 5 mitomycin studies, 2 of 4 doxorubicin studies, and 6 of 10 thiotepa studies; and a significant reduction in tumor progression was documented in 3 of 3 BCG studies, 0 of 2 mitomycin studies, 0 of 2 doxorubicin studies, and 0 of 3 thiotepa studies. Among these agents, BCG is the only one shown to result in a survival advantage over transurethral resection alone. (Herr et al, J Clin Oncol 1995, 13, 1404-8; Sarosdy & Lamm, J Urol 1989, 142, 719-22; Catalona et al, J Urol 1987, 137, 220-4; De Jager et al, Urology 1991, 38, 507-13; Herr et al, J Urol 1992, 147, 1020-3).

The mechanism of action of BCG in treatment of bladder cancer is unknown. However, the available evidence suggests that intravesical BCG is a form of immunotherapy. Intravesical BCG appears to induce tumor regression through a number of specific and non-specific actions. It promotes a local inflammatory reaction with histiocytic and leukocytic infiltration in the urinary bladder that is apparently associated with an elimination or reduction of superficial cancerous lesions.

Although BCG treatment of bladder cancer is efficient, there remain approximately 40% of patients for whom such treatment does not result in disappearance of the cancer. There is therefore a need in the art for more efficacious therapy of bladder cancer.

HPV Infection

Human papilloma virus (HPV) infections of the urogenital tract represent the most often sexually transmitted viral disease in humans. HPV is a double stranded DNA virus and with the recent developed molecular biological techniques, more than 55 different HPV types have been recognized. HPV is associated with a wide spectrum of clinical states including condylomata acuminata, latent and subclinical infection, and Bowen's disease. Subclinical infections gain more importance as they are believed to cause intraepithelial neoplasia, based on the frequent detection of HPV DNA in invasive carcinomas, especially in urogenital region. A significant risk for the development of an invasive cancer is ascribed to the infections by HPV types 1 6, 1 8 and 33.

The most prevalent HPV types causing condylomata acuminata are type 6 and 11. Condylomata acuminata are visible, multifocal, multicentric and multiform lesions. Predilection sites are penis, scrotum, perineum, urethra, perianal regions, intertriginous zones, and oral mucosa. In uncircumcised men the frenulurn, the coronary sulcus and the inner aspect of the foreskin are most often afflicted, whereas in circumcised patients the shaft of the penis is involved. Genital warts are of great psychological and cosmetic relevance representing a major hindrance to sexual performance.

Treatment options include surgical methods like excision, electrocautery, cryosurgery or laser vaporization. It has been shown in molecular hybridization studies that HPV DNA sequences exist in adjacent normal tissue after carbon dioxide laser removal of genital warts. These findings and the well known high recurrence rates after initial treatment demonstrate the need for adjuvant therapy to eradicate invisible disease.

Therapeutic results with local application of cytotoxic agents, for example, 5-fluorouracil and podophyllin/podophyllotoxin. More recently, it been suggested that treatment with a mycobacterial antigen such as attenuated BCG composition may be efficacious against HPV-related disease (PCT patent publication no. WO9955347, the disclosure of which is incorporated by reference). Despite the availability of therapeutic agents, treatment of HPV related disease to date remains unsatisfactory in its efficacy and side effects.

### SUMMARY

The present invention now discloses particular compositions and methods that can be used to efficiently treat a tumor, particularly a carcinoma or adenocarcinoma, and preferably a bladder cancer, in a subject. The invention also provides compositions and methods for the treatment of a viral infection, preferably an HPV infection, and conditions associated therewith such as cell proliferative disorders.

In one aspect, the inventors have provided that administration of a γδ T cell activating compound can enhance the effects of a locally administered immunomodulatory composition (IMC) or immunogenic composition (IC), regardless of whether the IMC or IC are γδ T cell activating or a non-γδ T cell activating compounds. Thus, in one aspect the invention provides a method for enhancing the effect of a locally administered immunomodulatory composition (IMC) or immunogenic composition (IC) in a mammal, the method comprising administering to the mammal a γδ T cell activating compound. In another aspect the invention encompasses a method for killing or inhibiting a proliferating cell, a tumor cell or an infected cell in a mammal, the method comprising administering to the mammal an immunomodulatory composition (IMC) or immunogenic composition (IC) locally to a site of disease, and administering a γδ T cell activating compound. These methods can be used advantageously for the treatment of mammals, particularly humans. Thus, in one embodiment, the present invention provides a method for treatment which involves administering an immunomodulatory composition (IMC) or immunogenic composition (IC) locally to a site of disease, and administering a γδ T cell activating compound.

The invention further discloses the use of an IMC or IC composition for the manufacture of a pharmaceutical composition or medicament, wherein said pharmaceutical composition or medicament is used or administered in combination with a γδ T cell activator. Likewise, the invention encompasses the use of a γδ T cell activator for the manufacture of a pharmaceutical composition or medicament, wherein said pharmaceutical composition or medicament is used or administered in combination with an IMC or IC composition. The invention further discloses the use of an IMC or IC composition and a γδ T cell activator for the manufacture of a pharmaceutical composition or medicament. Most preferably, the IMC or IC is for local administration to a site of disease. Also encompassed are related pharmaceutical compositions and kits comprising such compositions.

Preferably the γδ T cell activating compound will be administered by a route or to a site other than the site of disease to which the IMC or IC is administered. Most preferably the γδ T cell activating compound is administered by a method other than local administration to a disease site. The latter method of treatment comprising local and non-local administration can have beneficial effects, particularly when the locally-administered component is delivered intravesically or to skin, for example for treatment of bladder cancer, HPV infection, cell proliferative conditions such as skin disorders and external genital warts, and actinic keratosis and skin tumors, particularly non-melanoma skin cancers such as superficial basal cell carcinoma (BCC), or intra-tumorally, for the treatment of solid tumors. The treatments (and pharmaceutical compositions) of the present invention can particularly advantageously be used in the treatment of proliferative disorders, tumors, solid tumors, carcinomas, bladder cancer, HPV infection, cell proliferative conditions such as skin disorders, external genital warts, and actinic keratosis and skin tumors, particularly non-melanoma skin cancers such as superficial basal cell carcinoma (BCC).

In another aspect, the method provides that administration of a γδ T cell activating compound locally to a site of disease can have beneficial effects, particularly when delivered intravesically or to skin, for example for treatment of bladder cancer, HPV infection, cell proliferative conditions such as skin disorders and external genital warts, and actinic keratosis and skin tumors, particularly non-melanoma skin cancers such as superficial basal cell carcinoma (BCC). Thus in one embodiment, the invention provides a method comprising administering locally to a site of disease a composition capable of recruiting or preferably regulating γδ T cell activity or most preferably activating a γδ T cell. Preferably the composition comprises a γδ T cell activating compound, most preferably the compound selected from the group of: a compound capable of selectively activating a γδ T cell, a compound capable of activating a γδ T cell in a substantially pure culture of γδ T cells, and a compound of Formulas I to XVI.

The immunomodulatory composition (IMC) can generally be any agent that modulates one or more aspects of the immune system, for example by stimulating certain aspects of the immune system, or by suppressing certain other aspects, as further described herein. An immunogenic composition (IC) may be any agent capable of eliciting a humoral or cellular immune response, or both, when administered to an animal having an immune system. An IMC or IC may be, for example, capable of recruiting or preferably regulating the activity of, including but not limited to regulating cytokine production, activating or inhibiting, directly or indirectly, any type of immune cell, including for example activating γδ T cells or modulating an activity of γδ T cells, or modulating the activity of, particularly maturation of, dendritic cells. Thus, in certain embodiments an IMC or IC can also be a γδ T cell activating compound. In the context of the present invention, when two compositions are administered to an individual, they may be either the same or different compositions. In other embodiments, the IMC or IC can be any other suitable compound; an IMC may be for example a cytokine such as IL-2, IL-12, IL15, IL-21 or an agonist of a toll-like receptor (TLR), such as TLR2, TLR3, TLR4, TLR6, TLR7 or TLR9, or other agents described herein. Preferred ICs are polypeptide antigens, particularly microbial or tumor antigens, or a killed or attenuated pathogen, microorganism or parasite such as viruses or bacterial strains. A number of such agents are further described herein. Examples of routes of local administration to a site of disease can include but are not limited to dermal and intradermal, intravesical administration (e.g. bladder cancer), or generally intra-tumoral administration (e.g. solid tumors).

The γδ T cell activating compounds described herein can be any suitable γδ T cell activating compound. Such a compound can be prepared for use in local or non-local (to a site of disease) administration, including a range of γδ T cell activating compounds described herein.

One example of a type of composition that may be considered both an IMC and IC are mycobacterial antigens, of which several compositions are currently approved for human therapy for local administration. Administration of mycobacterial antigens may lead to activation of γδ T cells and therefore represent an example of the invention where the IC or IMC activates γδ T cells. In one specific example of the invention, a mycobacterial antigen is administered locally to a site of disease (e.g. intravesically, or to skin in the case of genital warts or HPV infection), and in conjunction with this local administration a γδ T cell activating compound that stimulates the proliferation and/or biological activity of γδ T cells is administered to the patient by a non-local route, preferably systemically, most preferably by intravenous or intramuscular administration. The γδ T cell activating compound administered systemically can be the same compound as the γδ T cell activating compound administered locally, or can be a different compound. However, when a mycobacterial antigen (for example a mycobacterial strain) is used as the locally administered compound, it will be preferably to use a different γδ T cell activating compound for systemic administration. A wide variety of preferred γδ T cell activating compounds for systemic administration, particularly synthetic and selective γδ T cell activating compounds are provided herein. Such compounds show little or no toxicity at doses required to activate γδ T cells. In this example, the combination therapy thereby preferably amplifies the γδ T cell-mediated effects of the composition that is administered locally.

In another example, the IMC compound or composition is an imidazoquinoline compound, and/or is an agonist of a toll-like receptor (TLR). At least one imidazoquinoline compound is currently approved for human therapy for local administration, and others in clinical development. A preferred example is Aldara™ (3M; imiquimod), formulated as a cream for dermal administration, for use in the treatment of superficial basal cell carcinoma, HPV infection, and also in testing for the treatment of cutaneous metastases of malignant melanoma (Bong et al, Dermatology 2002; 205:135-138). Another example is resiquimod (R-848; S-28463; 4-amino-2-ethoxymethyl-α, α-dimethyl-1H-imidazo[4,5-c]quinoline-1-ethanol). Resiquimod has been shown to induce endogenous production of alpha interferon (IFN-α), interleukin 12 (IL-12), tumor necrosis factor alpha, and other cytokines from peripheral blood mononuclear cells, monocytes, and dendritic cells (DCs). Resiquimod is about 100 times more potent in inducing cytokines in vitro and in vivo than the related imidazoquinoline imiquimod (Aldara R-837) (Sauder et al, (2003) Antimicrobial Agents and Chemotherapy, 47(12): 3846-3852). Resiquimod and imiquimod also differ in the cytokine induction profile: in peripheral blood mononuclear cell cultures, resiquimod induces larger amounts of IL-12 directly and larger amounts of IFN-γ indirectly. Resiquimod is also more effective in enhancing antigen presentation by DCs. Induction of these cytokines by both compound is thought to involve the Toll-like receptor (TLR) signaling pathway, particularly Toll-like receptor 7 (TLR7) and TLR8. In one specific example of the invention, a TLR agonist such as imiquimod or resiquimod is administered locally to a site of disease (e.g. dermally in the case of skin proliferative disorder or skin cancer, or genital warts or HPV infection), and in conjunction with this local administration a γδ T cell activating compound that stimulates the proliferation and/or biological activity of γδ T cells is administered to the patient by a non-local route, preferably systemically, most preferably by intravenous or intramuscular administration. Other TLR agonist compounds are known in the art and/or described in references cited herein, including but not limited to nucleic acid-based agonists such as CpG containing nucleic acids (TLR9 agonists) and double-stranded RNA (TLR3 agonists).

Generally, in preferred embodiments the locally-administered IMC or IC is administered in an amount effective to treat said disease when used in combination therapy with the second compound which is a γδ T cell activator. Preferably said γδ T cell activator is administered systemically, preferably by intravenous, subcutaneous or intramuscular injection. In a preferred aspect, the IMC comprises a compound capable of activating a γδ T cell, a cytokine, or a compound which is an agonist of a toll-like receptor (TLR). In preferred examples, the agonist of a TLR is an imidazoquinoline compound or analog or derivative thereof, or a mycobacterium antigen. Said disease is preferably a proliferative disorder, a carcinoma or a viral infection; preferred examples include respectively a bladder cancer, a skin tumor or cancer, or an HPV infection.

In another embodiment, the invention provides a method for the treatment of a disease comprising:
(a) administering to said subject a first γδ T cell activator compound in a quantity sufficient to stimulate γδ T cell activity; and
(b) administering to a subject locally at a site of disease, a second γδ T cell activator, said second γδ T cell activator being administered in a quantity effective to treat said disease when used in combination therapy with said first γδ T cell activator.

Said first and second γδ T cell activators may comprise the same compound or composition or may comprise different compounds or compositions. Preferably the second γδ T cell activator is a mycobacterium antigen, and preferably the first γδ T cell activator is a selective γδ T cell activator. Preferably the first γδ T cell activator is administered systemically, preferably by intravenous injection. Said disease is preferably a carcinoma or a viral infection; preferred examples include respectively a bladder cancer or an HPV infection.

In a further embodiment, the invention also provides that a selective γδ T cell activator can be used in combination with a mycobacterial antigen, preferably by administration via the same route, e.g. preferably intravesical administration or administration to skin. The invention thus discloses a method for treating bladder cancer or HPV infection in a patient comprising administering to a patient in need thereof an amount of a Mycobacterium antigen and a γδ T cell activator effective to treat said disease. Preferably a mycobacterial antigen and a selective γδ T cell activator are both administered at a site of disease (e.g. intravesicularly or to skin). The mycobacterial antigen and the selective γδ T cell activator can be administered at the same time or at different times, and can be provided in separate compositions or as a single composition. The invention thus also discloses a pharmaceutical composition comprising an IC or IMC and a γδ T cell activator at an effective dose to treat a carcinoma or viral infection, preferably wherein the IMC or IC is not interleukin-2. In another embodiment, the invention discloses a pharmaceutical composition comprising a Mycobacterium antigen and a γδ T cell activator, preferably at an effective dose to treat a carcinoma such as bladder cancer, urinary cancer or a viral infection such as an HPV infection. The invention thus also provides the use of a γδ T cell activator for the manufacture of a pharmaceutical composition for the treatment of bladder cancer, urinary cancer or a viral infection such as an HPV infection. The invention further discloses the use of a Mycobacterium antigen and a γδ T cell activator for the manufacture of a pharmaceutical composition for the treatment of bladder cancer. The invention also discloses a kit for the treatment of bladder cancer comprising a Mycobacterium antigen and a γδT cell activator.

Preferably, said Mycobacterium antigen is an antigen of Mycobacterium bovis. Alternatively, said Mycobacterium antigen is an antigen of Mycobacterium phlei. More preferably, said Mycobacterium antigen is an attenuated strain thereof. Still more preferably, said Mycobacterium antigen is an attenuated strain of Mycobacterium bovis (BCG). In a particular embodiment, said Mycobacterium antigen is mycobacterial cell wall, preferably complexed to Mycobacterium DNA.

In preferred embodiments, a γδ T cell activator is a selective γδ T cell activator capable of regulating the activity of a γδ T cell in a population of γδ T cell in culture, most preferably in a substantially pure population of γδ T cells, or in a population of γδ T cell clones. The γδ T cell activator is preferably capable of regulating the activity of a γδ T cell population of γδ T cell clones at millimolar concentration, preferably when the γδ T cell activator is present in culture at a concentration of less than 100 mM. Optionally a γδ T cell activator is capable of regulating the activity of a γδ T cell in a population of γδ T cell clones at millimolar concentration, preferably when the γδ T cell activator is present in culture at a concentration of less than 10 mM, or more preferably less than 1 mM. Regulating the activity of a γδ T cell can be assessed by any suitable means, preferably by assessing cytokine secretion, most preferably TNF-α secretion as described herein. Methods for obtaining a population of pure γδ T cell clones is described in Davodeau et al, (1993) J. Immunology 151(3): 1214-1223 and Moreau et al, (1986) J. Clin. Invest. 78:874, the disclosures of which are incorporated herein by reference. Preferably the activator is capable of causing at least a 20%, 50% or greater increase in the number of γδ T cells in culture, or more preferably at least a 2-fold increase in the number of γδ T cells in culture.

Preferably, said γδT cell activator is a composition comprising a compound of formula (I): 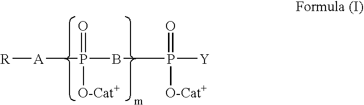
wherein Cat+ represents one (or several, identical or different) organic or mineral cation(s) (including proton);
m is an integer from 1 to 3;
B is O, NH, or any group capable to be hydrolyzed;
Y═O⁻Cat+, a C₁-C₃ alkyl group, a group -A-R, or a radical selected from the group consisting of a nucleoside, an oligonucleotide, a nucleic acid, an amino acid, a peptide, a protein, a monosaccharide, an oligosaccharide, a polysaccharide, a fatty acid, a simple lipid, a complex lipid, a folic acid, a tetrahydrofolic acid, a phosphoric acid, an inositol, a vitamin, a co-enzyme, a flavonoid, an aldehyde, an epoxyde and a halohydrin;
A is O, NH, CHF, CF₂ or CH₂; and,
R is a linear, branched, or cyclic, aromatic or not, saturated or unsaturated, C₁-C₅₀ hydrocarbon group, optionally interrupted by at least one heteroatom, wherein said hydrocarbon group comprises an alkyl, an alkylenyl, or an alkynyl, preferably an alkyl or an alkylene, which can be substituted by one or several substituents selected from the group consisting of: an alkyl, an alkylenyl, an alkynyl, an epoxyalkyl, an aryl, an heterocycle, an alkoxy, an acyl, an alcohol, a carboxylic group (—COOH), an ester, an amine, an amino group (—NH₂), an amide (—CONH₂), an imine, a nitrile, an hydroxyl (—OH), a aldehyde group (—CHO), an halogen, an halogenoalkyl, a thiol (—SH), a thioalkyl, a sulfone, a sulfoxide, and a combination thereof.

In a more preferred embodiment, said γδ T cell activator is a composition comprising a compound of formula (II): 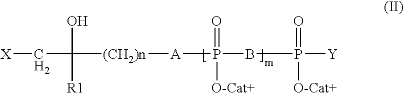
in which X is an halogen (preferably selected from I, Br and Cl), B is O or NH, m is an integer from 1 to 3, R1 is a methyl or ethyl group, Cat+ represents one (or several, identical or different) organic or mineral cation(s) (including the proton), and n is an integer from 2 to 20, A is O, NH, CHF, CF₂ or CH₂, and Y is O⁻Cat+, or a nucleoside. Still more preferably, said γδ T cell activator is selected from the group consisting of BrHPP, CBrHPP and epoxPP. Optionally, said γδ T cell activator is BrHPP. Alternatively, said γδ T cell activator is CBrHPP. Otherwise, said γδ T cell activator is epoxPP.

In an other more preferred embodiment, said γδ T cell activator is a composition comprising a compound of formula (XII): 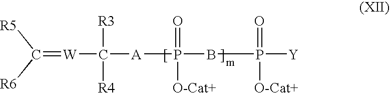
in which R₃, R₄, and R₅, identical or different, are a hydrogen or (C₁-C₃)alkyl group, W is —CH— or —N—, R₆ is an (C₂-C₃)acyl, an aldehyde, an (C₁-C₃)alcohol, or an (C₂-C₃)ester, Cat+ represents one (or several, identical or different) organic or mineral cation(s) (including the proton), B is O or NH, m is an integer from 1 to 3, A is O, NH, CHF, CF₂ or CH₂, and Y is O⁻Cat+, or a nucleoside. Still more preferably, said γδ T cell activator is selected from the group consisting of HDMAPP and CHDMAPP. In a first most preferred embodiment, said γδ T cell activator is HDMAPP. In a second one, said γδ T cell activator is CHDMAPP.

In a preferred embodiment, the (a) the IMC or IC, or the Mycobacterium antigen and (b) the γδT cell activator are administered within about one week, 3 days, or more preferably 48 hours, or about 24 hours of one another. Optionally said the IMC or IC, or the Mycobacterium antigen and γδT cell activator are administered simultaneously, or within 6 hours of one another. Suitable treatment regimens may specify that the γδT cell activator can be administered before or after said the IMC or IC, or the Mycobacterium antigen. In the case of combined use of Mycobacterium antigen and γδT cell activator, the compounds can be administered by the same routes. Alternatively, said Mycobacterium antigen and γδT cell activator can be administered by different routes. In a most preferred embodiment, the IMC or IC, or the Mycobacterial antigen is administered locally to a site of disease and a γδT cell activator is administered by a non-local route, preferably by systemic administration.

Preferably, for the treatment of bladder carcinoma, a Mycobacterium antigen is administered intravesicularly into the bladder. Preferably, said Mycobacterium antigen is administered after a transurethal resection, still more preferably 1 or 2 weeks following transurethal resection. In a preferred embodiment, said bladder cancer is a stage 0 bladder cancer. More preferably, said stage 0 bladder cancer is a non-invasive papillomary carcinoma (TaT1) or a carcinoma in situ (CIS).

### DESCRIPTION OF THE FIGURES

FIG. 1 shows the synthesis scheme for the compound referred to herein as CHDMAPP, the complete synthesis of which is described in Example 2.

### DETAILED DESCRIPTION

Definitions

Where “comprising” is used, this can preferably be replaced by “consisting essentially of”, more preferably by “consisting of”.

As used in the specification, “a” or “an” may mean one or more. As used in the claim(s), when used in conjunction with the word “comprising”, the words “a” or “an” may mean one or more than one. As used herein “another” may mean at least a second or more.

Where hereinbefore and hereinafter numerical terms are used, they are meant to include the numbers representing the upper and lower limits. For example, “between 1 and 3” stands for a range “from and including 1 up to and including 3”, and “in the range from 1 to 3” would stand for “from and including 1 up to and including 3”. The same is true where instead of numbers (e.g. 3) words denoting numbers are used (e.g. “three”).

“Weekly” stands for “about once a week” (meaning that more than one treatment is made with an interval of about one week between treatments), the about here preferably meaning +/−1 day (that is, translating into “every 6 to 8 days”); most preferably, “weekly” stands for “once every 7 days”.

“3-weekly” or “three-weekly” stands for “about once every three weeks” (meaning that more than one treatment is made with an interval of about three weeks between treatments), the about here preferably meaning +/−3 days (that is, translating into every 18 to 24 days); most preferably, “weekly” stands for “once every 21 days” (=every third week).

The term “about” or “approximately” usually means within 20%, more preferably within 10%, and most preferably still within 5% of a given value or range. Alternatively, especially in biological systems (e.g., when measuring an immune response), the term “about” means within about a log (i.e., an order of magnitude) preferably within a factor of two of a given value.

Whenever within this whole specification “treatment of a tumor” or the like is mentioned with reference to the compositions according the present invention, essentially a locally delivered immunomodulatory compound (IMC) or immunogenic compound (IC), preferably an antigen such a mycobacterial antigen or a TLR agonist or imidazoquinoline compound, and a γδT cell activator, there is meant:

a) a method of treatment (=for treating) of a tumor, said method comprising the step of administering (for at least one treatment) an ICM or IC and a γδT cell activator, (preferably each in a pharmaceutically acceptable carrier material) to a mammal, especially a human, in need of such treatment, in a dose that allows for the treatment of said tumor (=a therapeutically effective amount), preferably in a dose (amount) as specified to be preferred hereinabove and hereinbelow;

b) the use of an ICM or IC and a γδT cell activator for the treatment of a tumor; or an ICM or IC and a γδT cell activator, for use in said treatment (especially in a human);

c) the use of an ICM or IC and a γδT cell activator, for the manufacture of a pharmaceutical preparation(s) for the treatment of a tumor; and/or

d) a pharmaceutical preparation comprising a dose of an ICM or IC and a dose of a γδT cell activator that is appropriate for the treatment of a tumor; or any combination of a), b), c) and d), in accordance with the subject matter allowable for patenting in a country where this application is filed;

e) a method of using an ICM or IC and a γδT cell activator for the manufacture of a pharmaceutical preparation(s) for the treatment of a tumor, comprising admixing each said ICM or IC and said γδT cell activator(s) with a pharmaceutically acceptable carrier, preferably in separate containers to be used in combination therapy. It will be appreciated that references for example to treatment of a particular condition mentioned herein, for example cancer, proliferative disorder, carcinoma, bladder cancer, urinary cancer, skin proliferative disorder, basal cell carcinoma, genital warts, actinic keratosis, viral infection or HPV infection, can be substituted in the above definition in the same way as the term tumor, and such will be understood according to the same above definition as exemplified for tumor.

Within the context of the present invention, the expressions “stimulating the activity of γδ T cells”, “activating γδ T cells” and “regulating the activity of γδ T cells” designate causing or favoring an increase in the number and/or biological activity of such cells in a subject. Stimulating and regulating thus each include without limitation modulating (e.g., stimulating) expansion of such cells in a subject and/or, for instance, triggering of cytokine secretion (e.g., TNFα or IFNγ). γδ T cells normally represent between about 1-10% of total circulating lymphocytes in a healthy adult human subject. The present invention can be used to significantly increase the γδ T cells population in a subject, particularly to reach at least 30% of total circulating lymphocytes, typically 40%, more preferably at least 50% or 60%, or from 50%-90%. Regulating also includes, in addition or in the alternative, modulating the biological activity of γδ T cells in a subject, particularly their cytolytic activity or their cytokine-secretion activity. The invention defines novel conditions and strategies for increasing the biological activity of γδ T cells towards target cells.

As used herein, the term “immunogenic” means that an agent is capable of eliciting a humoral or cellular immune response, and preferably both. An immunogenic entity is also antigenic. An immunogenic composition is a composition that elicits a humoral or cellular immune response, or both, when administered to an animal having an immune system.

The term “antigen” refers to any agent (e.g., protein, peptide, lipid, polysaccharide, glycoprotein, glycolipid, nucleic acid or any combination of any of the foregoing) that, when introduced into a host, animal or human, having an immune system (directly or upon expression as in, e.g., DNA vaccines), is recognized by the immune system of the host and is capable of eliciting an immune response. As defined herein, the antigen-induced immune response can be humoral or cell-mediated, or both. An agent is termed “antigenic” when it is capable of or comprises a component capable of specifically interacting with an antigen recognition molecule of the immune system, such as an immunoglobulin (antibody) or T cell antigen receptor (TCR). Examples of preferred antigens are “surface antigens”, i.e., expressed naturally on the surface of a pathogen, or the surface of an infected cell, or the surface of a tumor cell. A molecule that is antigenic need not be itself immunogenic, i.e., capable of eliciting an immune response without an adjuvant or carrier. An antigen may be “species-specific”, referring to an antigen that is only present in or derived from a particular species.

The terms “vector”, “cloning vector”, and “expression vector” mean the vehicle by which a DNA or RNA sequence (e.g., a foreign gene) can be introduced into a host cell, so as to transform the host and promote expression (e.g., transcription and/or translation) of the introduced sequence. Vectors include plasmids, phages, viruses, etc.

In accordance with the present invention there may be employed conventional molecular biology, microbiology, and recombinant DNA techniques within the skill of the art. Such techniques are well-known and are explained fully in the literature. See, e.g., Sambrook, Fritsch and Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning. A Practical Approach, Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. Perbal, A Practical Guide To Molecular Cloning (1984); F. M. Ausubel et al. (eds.), Current Protocols in Molecular Biology, John Wiley & Sons, Inc. (1994).

A “nucleic acid molecule” (or alternatively “nucleic acid”) refers to the phosphate ester polymeric form of ribonucleosides (adenosine, guanosine, uridine, or cytidine: “RNA molecules”) or deoxyribonucleosides (deoxyadenosine, deoxyguanosine, deoxythymidine, or deoxycytidine: “DNA molecules”), or any phosphoester analogs thereof, such as phosphorothioates and thioesters, in either single stranded form, or a double-stranded helix. Oligonucleotides (having fewer than 100 nucleotide constituent units) or polynucleotides are included within the defined term as well as double stranded DNA-DNA, DNA-RNA, and RNA-RNA helices. This term, for instance, includes double-stranded DNA found, inter alia, in linear (e.g., restriction fragments) or circular DNA molecules, plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA). A “recombinant DNA molecule” is a DNA molecule that has undergone a molecular biological manipulation.

As used herein, the term “polypeptide” refers to an amino acid-based polymer, which can be encoded by a nucleic acid or prepared synthetically. Polypeptides can be proteins, protein fragments, chimeric proteins, etc. Generally, the term “protein” refers to a polypeptide expressed endogenously in a cell. Generally, a DNA sequence encoding a particular protein or enzyme is “transcribed” into a corresponding sequence of mRNA. The mRNA sequence is, in turn, “translated” into the sequence of amino acids which form a protein. An “amino acid sequence” is any chain of two or more amino acids. The term “peptide” is usually used for amino acid-based polymers having fewer than 100 amino acid constituent units, whereas the term “polypeptide” is reserved for polymers having at least 100 such units. Herein, however, “polypeptide” will be the generic term.

As used herein, the terms “in combination” or “combination therapy”, used interchangeably, refer to the situation where two or more therapeutic agents affect the treatment or prevention of the same disease. The use of the term “in combination” does not restrict the order in which therapies (e.g., prophylactic or therapeutic agents) are administered to a subject with the disease. A first therapy can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject with a disease.

When one or more agents are used in combination in a therapeutic regimen, there is no requirement for the combined results to be additive of the effects observed when each treatment is conducted separately. Although at least additive effects are generally desirable, any increased effect, for example an anti-cancer effect, above one of the single therapies would be of benefit. Also, there is no particular requirement for the combined treatment to exhibit synergistic effects, although this is certainly possible and advantageous.

In one aspect, the present invention concerns a method for inhibiting the growth of cancer cells in the urinary bladder of a mammal having a bladder cancer comprising administering a Mycobacterium antigen and a γδT cell activator.

In one aspect, the present invention also concerns a method of preventing, or treating a carcinoma or viral infection, preferably a urinary or bladder cancer or an HPV infection in a mammal comprising administering a Mycobacterium antigen, and a γδT cell activator.

In one aspect, the present invention further concerns a pharmaceutical composition comprising a Mycobacterium antigen, and a γδT cell activator and the use thereof for treatment or prevention of carcinoma or viral infection, preferably a urinary or bladder cancer or an HPV infection. Optionally, said composition is prepared for separate administration of said Mycobacterium antigen and said γδT cell activator. Optionally, said composition further comprises a cytokine. Optionally, said composition also comprises an additional agent active against carcinoma or viral infection, preferably a urinary or bladder cancer or an HPV infection. Such agent includes, but are not limited to, drugs, immunostimulants, antigens, antibodies, vaccines, radiation and chemotherapeutic, genetic, biologically engineered and chemically synthesized agents, and agents that target cell death molecules for activation or inactivation and that inhibit proliferation of and induce apoptosis in responsive cells.

The present invention concerns a pharmaceutical composition comprising a Mycobacterium antigen, and a γδT cell activator for use as a medicament. More particularly, the invention concerns the use of a Mycobacterium antigen and a γδT cell activator for the manufacture of a medicament for the treatment of carcinoma or viral infection, preferably a urinary or bladder cancer or an HPV infection.

The invention also contemplates the methods and the compositions comprising several Mycobacterium antigens and/or several γδ T cell activators.

In a first embodiment, said Mycobacterium antigen and said γδT cell activator are administered simultaneously to said mammal. More particularly, a pharmaceutical composition comprising said Mycobacterium antigen and said γδT cell activator is administered to said mammal. In preferred aspects, said Mycobacterium antigen and said γδT cell activator can be administered by separately and are administered by different routes of administration, for example the mycobacterial antigen is administered locally at a disease site and the γδT cell activator is administered systemically, preferably by intravenous (iv) route. Said Mycobacterium antigen can be administered to said mammal before or after said γδT cell activator.

In further preferred aspects, the methods may comprise further administering a cytokine. Said cytokine is capable of increasing the expansion of a γδ T cell population treated with a γδ T cell activator compound. A preferred cytokine is an interleukin-2 polypeptide (e.g., Research Diagnostics, NJ, #RDI-202). For example, cytokines for use in accordance with the invention and regimens for their administration are described is PCT patent publication no WO 01/56387, the disclosure of which is incorporated herein by reference.

The present invention more particularly concerns a freeze-dried (lyophilized) pharmaceutical composition comprising a Mycobacterium antigen, and a γδT cell activator. Preferably, the pharmaceutical composition according to the present invention is administered as an aqueous suspension. For administration in an aqueous carrier, the pharmaceutical composition according to the present invention is suspended in a pharmaceutically acceptable buffer including, but not limited to, saline and phosphate buffered saline (PBS) and is either asceptically processed or terminally sterilized. For example, freeze-dried (lyophilized) pharmaceutical composition according to the present invention may be stored in sealed ampoules or vials requiring only the addition of a carrier, for example sterile water, immediately prior to use.

Therefore, the present invention also concerns a kit comprising at least one container and a pharmaceutical composition according to the present invention. Preferably, containers are sealed ampoules or vials. Optionally, the kit can comprise a syringe. The kit can comprise a container comprising both Mycobacterium antigen, and γδT cell activator. The kit can also comprise a container comprising the Mycobacterium antigen and an other one comprising the γδT cell activator. Preferably, the pharmaceutical composition is freeze-dried (lyophilized).

Preferably, the pharmaceutical composition according to the present invention, in combination with a pharmaceutically acceptable carrier, is administered to a mammal locally to a site of disease in a dosage effective to treat the carcinoma. For example, in bladder cancer, local administration refers to administration into the bladder

Routes of administration for the γδ T cell activator, the IMC and the IC compounds and mycobacterium antigens, include, but are not limited to, oral, dermal, subcutaneous, percutaneous, intramuscular, intraperitoneal, intravenous, intradermal, intrathecal, intralesional, intratumoral, intrabladder, intra-vaginal, intraocular, intrarectal, intrapulmonary, intraspinal, transdermal, subdermal, placement within cavities of the body, nasal inhalation, pulmonary inhalation, impression into skin and electrocorporation.

Any suitable method for administering the mycobacterial antigen can be used, depending on the disease. For example, for bladder cancer, preferred methods are as follows. The Mycobacterium antigen is administered by instillation into the urinary bladder by, but not limited to, a urinary tract catheter. Other methods for instilling the pharmaceutical composition according to the present invention into the urinary bladder are known to those skilled in the art. The γδT cell activator is provided by systemic administration, preferably by intravenous infusion or intramuscular injection. The bladder cancer is preferably a stage 0 bladder cancer. More preferably, the bladder cancer is a non-invasive papillomary carcinoma (TaT1) or a carcinoma in situ (CIS). More particularly, the methods of treatment and the pharmaceutical compositions according to the present invention are well adapted for the primary treatment of CIS of the bladder (after transurethial resection) either with or without associated papillary tumors, the secondary treatment of CIS of the bladder in patients treated with other intravesical agents who have relapsed or failed to respond, and the primary or secondary treatment of CIS in patients who have contraindications to radical surgery. Furthermore, these methods and compositions are also well adapted for the adjuvant treatment following transurethal resection of stage Ta or T1 papillary tumors of the bladder, which are at high risk of recurrence. In a preferred aspect of the present invention, the pharmaceutical compositions according to the present invention are administered to a subject having a bladder cancer after a step of transurethial resection. In a preferred embodiment, the treatment is administered 7-15 days after the transurethial resection.

In other examples, it will be appreciated that any suitable composition comprising an antigen can be used in the same way as or in place of the Mycobacterium antigen in the methods described herein. In particular, examples of composition suitable for such use include those comprising a killed, inactivated or attenuated pathogen, microorganism or parasite. In other aspects, a composition comprising an antigen preferably comprises an enriched or purified polypeptide, lipid, polysaccharide, glycoprotein, glycolipid or nucleic acid antigen. Preferably said composition comprises at least 1, 2, 3, 4, 5, 10 or 15 distinct antigens, for example at least 1, 2, 3, 4, 5, 10 or 15 distinct polypeptides, or nucleic acids encoding such polypeptides. Further examples of compositions are provided herein.

In yet further embodiments, it will be appreciated that any suitable composition comprising an antigen can be used as the IC component in any of the methods of the invention. Most preferably the composition comprising an antigen is administered intra-tumorally for the treatment of a tumor or cancer. In particular, examples of composition suitable for use include those comprising a killed, inactivated or attenuated pathogen, microorganism or parasite. In other aspect, a composition comprising an antigen preferably comprises an enriched or purified polypeptide, lipid, polysaccharide, glycoprotein, glycolipid or nucleic acid antigen. Preferably said composition comprises at least 1, 2, 3, 4, 5, 10 or 15 distinct antigens, for example at least 1, 2, 3, 4, 5, 10 or 15 distinct polypeptides, or nuclei acids encoding such polypeptides. In other preferred examples, the present invention concerns a method for inhibiting the growth of proliferating cells, preferably tumor or cancer cells, in a mammal, comprising (a) administering an antigen to the mammal locally to a site of disease, and (b) administering a γδT cell activator to the mammal. In preferred aspects step (a) comprises administering a nucleic acid encoding an antigen or administering a polypeptide antigen. Optionally the γδT cell activator of step (b) is administered by an administration route other than intra-tumoral administration. As mentioned, most preferably the antigen is administered intra-tumorally. Any suitable solid tumor may be treated by this manner, including for example prostate, breast, colorectal, lung, pancreatic, renal or melanoma cancers.

Particularly preferred are tumor antigens. For example, PCT patent application no. W0 97/18837 disclose methods to produce gram-negative bacteria having non-pyrogenic Lipid A or LPS. Preferred bacteria are capable of eliciting an immune response in an individual. A preferred live bacterial vaccine must be immunogenic so that it elicits an immune response; however, the vaccine must not be capable of excessive growth in vivo which might result in adverse reactions. For example, some suitable bacterial vaccine vectors are temperature sensitive, having minimal replicative ability at normal physiological ranges of body temperature. Other examples of suitable compositions are further described herein.

In other preferred embodiments, a nucleic acid encoding an immunomodulatory polypeptide, or a vector comprising such nucleic acid, is used as the IMC component in any of the methods of the invention. One preferred example is a cytokine polypeptide, preferably a recombinant, purified or isolated polypeptide, or a fragment, variant or derivative thereof, selected form the group consisting of IFNγ, IL-1α, IL-1β, IL-2, IL-4, IL-5, IL-6, IL-7, IL-8, IL-9, IL-10, IL-11, IL-12, IL-13, IL-15, IL-18, IL-21, IL-23, IL-24, IL-27, IL-28a, IL-28b and IL-29. Other preferred examples include but are not limited to a nucleic acid encoding an antigen polypeptide, and a vector comprising a nucleic acid sequence encoding a cytokine.

Most preferably the polypeptide or nucleic acid is administered intra-tumorally for the treatment of a tumor or cancer.

In other preferred examples, the present invention concerns a method for inhibiting the growth of proliferating cells, preferably tumor or cancer cells, in a mammal, comprising (a) administering an immunogenic compound (IC) to the mammal locally to a site of disease, and (b) administering a γδT cell activator to the mammal. In one embodiment, the IC is administered to skin or intra-tumorally. In another preferred aspect step (a) comprises administering a nucleic acid encoding an IC or administering a polypeptide IC, preferably wherein the IC is a cytokine or an antigen. Optionally the γδT cell activator of step (b) is administered by an administration route other than locally to said site of disease, or other than by intra-tumoral administration. In one aspect, a composition comprising a nucleic acid encoding an antigen is administered intra-tumorally. Any suitable solid tumor may be treated by this manner, including for example prostate, breast, colorectal, lung, pancreatic, renal or melanoma cancers. Particularly preferred are nucleic acids encoding viral or tumor antigens or cytokines, or polypeptide cytokines or viral or tumor antigens.

When nucleic acids are administered, the nucleic acids can be prepared in any suitable manner. For example, the nucleic acids may be formulated for delivery as “naked” DNA, or are preferably inserted into a recombinant vector, for example an adenoviral vector (Ad), adeno-associated viral vector (AAV), vaccinia or poxvirus vectors. For example, PCT publication no. WO 98/04705 describes recombinant vectors containing inserted DNA fragments coding for a polypeptide from an early region and a polypeptide from a late region of a papillomavirus for treating or preventing a papillomavirus infection or tumour. PCT publication no. WO 01/18035 describes recombinant vectors encoding polypeptides derived from the MUC-1 polypeptide which are able to activate Cytotoxic T Lymphocyte (CTL) response. U.S. Pat. Nos. 6,007,806 and 5,744,133 describe recombinant vaccinia virus vectors comprising a heterologous DNA sequence which codes at least for the essential region of a tumor specific protein. PCT publication no. WO 86/07610 describes recombinant poxvirus vectors comprising a nucleic acid sequence encoding a human IL-2 protein. PCT publication no. WO 95/09241 describes a viral vector comprising a nucleic acid encoding for all or part of an immune and/or inflammatory response modulating polypeptide, for the treatment of cancers in mammals, including a poxvirus-derived viral vector comprising a nucleic acid coding for a cytokine such as IL-2, IL-4, IL-5, IL-6 or IL-7, gamma interferon, colony-stimulating factor or type ‘beta’ tumour necrosis factor. Other examples of suitable cytokines and antigens that can be encoded by the nucleic acids are further described herein. In a preferred example, the present invention concerns a method for inhibiting the growth of proliferating cells, preferably tumor or cancer cells, in a mammal, comprising (a) administering to the mammal, intra-tumorally, a composition comprising a nucleic acid encoding a cytokine, and (b) administering to the mammal a γδT cell activator by an administration route other than intra-tumoral administration. In a preferred example, the nucleic acid encodes IL-2, IL-12, IL-15 or IL-21 or a fragment, variant or derivative thereof.

In other preferred examples, the present invention concerns a method for inhibiting the growth of proliferating cells, preferably tumor or cancer cells, in a mammal, comprising administering to the mammal a TLR agonist, particularly a imidazoquinoline compound, and a γδT cell activator. In other preferred examples, the present invention concerns a method for treating or preventing a viral or bacterial infection, an HPV infection, cell proliferative conditions such as skin disorders and external genital warts, and actinic keratosis and skin tumors, particularly non-melanoma skin cancers such as superficial basal cell carcinoma (BCC), in a mammal, comprising administering to the mammal a TLR agonist, particularly a imidazoquinoline compound, and a γδT cell activator. In preferred examples, the immunomodulatory compound is administered to skin.

The present invention further concerns a pharmaceutical composition or a kit comprising an immunomodulatory compound (IMC), preferably a TLR agonist or imidazoquinoline compound, and a γδT cell activator, and the use thereof for treatment or prevention of disease. Optionally, said composition further comprises a cytokine. Optionally, said composition also comprises an additional agent active against the particular disease. Such agent includes, but are not limited to, drugs, immunostimulants, antigens, antibodies, vaccines, radiation and chemotherapeutic, genetic, biologically engineered and chemically synthesized agents, and agents that target cell death molecules for activation or inactivation and that inhibit proliferation of and induce apoptosis in responsive cells. The present invention concerns a pharmaceutical composition comprising an immunomodulatory compound (IMC), preferably a TLR agonist or imidazoquinoline compound, and a γδT cell activator for use as a medicament, preferably in separate containers. More particularly, the invention concerns the use of an immunostimulatory compound (IMC), preferably a TLR agonist or imidazoquinoline compound, and a γδT cell activator for the manufacture of a medicament for the treatment or prevention of a viral or bacterial infection, an HPV infection, cell proliferative conditions such as skin disorders and external genital warts, and actinic keratosis and skin tumors, particularly non-melanoma skin cancers such as superficial basal cell carcinoma (BCC).

In a first exemplary embodiment, an imidazoquinoline compound imiquimod (Aldara™) cream is administered dermally to said mammal at a site of genital warts or basal cell carcinoma. The same day, preferably within 6 hours before or after imiquimod application, the γδT cell activator is administered by intravenous (iv) or intramuscular route. In further preferred aspects, the methods may comprise further administering a cytokine. Said cytokine is capable of increasing the expansion of a γ [symbol] T cell population treated with a γ [symbol] T cell activator compound. A preferred cytokine is an interleukin-2 polypeptide; examples of low dose cytokine regimens for use in accordance with the invention are described is PCT patent publication no WO 01/56387, the disclosure of which is incorporated herein by reference. Routes of administration include, but are not limited to, oral, dermal, subcutaneous, percutaneous, intramuscular, intraperitoneal, intravenous, intradermal (PCT patent publication no WO 04/020014), intrathecal, intralesional, intratumoral, intrabladder, intra-vaginal, intraocular, intrarectal, intrapulmonary, intraspinal, transdermal, subdermal, placement within cavities of the body, nasal inhalation, pulmonary inhalation, impression into skin and electroporation.

It will be appreciated that the immunomodulatory or immunogenic composition and γδ T cell activating compound can therefore be advantageously used in a combination therapy. The immunomodulatory or immunogenic composition can be administered in a therapeutically effective amount, simultaneously in one composition, or simultaneously in different compositions, or sequentially. For the sequential administration of a first and second composition to be considered a combination therapy, however, the first and second compositions must be administered separated by a time interval that still permits the first composition to be used during a treatment cycle of the second composition, or that permits the first composition to show enhanced activity, particularly therapeutic activity, when compared with the single components alone. For example, when the immunomodulatory or immunogenic composition is a mycobacterium, the mycobacterium and the γδ T cell activating compound are administered within a week, within 5, 4, or 3 days of one another, or within 48 or 24 hours of one another, preferably within 6 hours of each other, and most preferably simultaneously.

Immunomodulatory Compounds (IMC)

An immunomodulatory compound for use according to the invention is generally any suitable compound that can be administered locally to a site of diseases. As used herein, the term “immunomodulatory compound”, “immunomodulatory composition” or IMC, and variations thereof, including but not limited to immunomodulant or immunomodulatory drug, refer to a compound that modulates a subject's immune system. In particular, an immunomodulatory compound is a compound that alters the ability of a subject's immune system to respond to one or more foreign antigens. In a specific embodiment, an immunomodulatory compound is a compound that shifts one aspect of a subject's immune response. In a preferred embodiment of the invention, an immunomodulatory compound is a compound that inhibits or reduces a subject's immune system (i.e., an immunosuppressant (compound). In one example, an immunomodulatory compound is a compound that enhances or increases a subject's immune system and/or enhances or increases the ability of a subject's immune system to respond to one or more foreign antigens; such compound may also be referred to herein as an immunostimulatory compound.

Examples of immunomodulatory compounds include B7 molecules (B7-1, B7-2, variants thereof, and fragments thereof) (see, e.g., Adv Exp Med Biol. 2000; 465:381-90), ICOS, and OX40 (see Coyle et al., Springer Semin Immunopathol. 2004 February; 25 (3-4):349-59), a negative T cell regulator such as an antibody against CTLA4 or against another negative immune cell regulator, such as BTLA and PD-1, and cytokines and growth factors including but not limited to IFNγ, IL-1α, IL-1β, IL-2, IL-4, IL-5, IL-6, IL-7, IL-8, IL-9, IL-10, IL-11, IL-12, IL-13, IL-15, IL-18, IL-21, IL-23, IL-24, IL-27, IL-28a, IL-28b, IL-29, KGF, TGFβ, M-CSF, G-CSF, TNFβ, LAF, TCGF, BCGF, TRF, BAF, BDG, MP, LIF, OSM, TMF, PDGF, IPN-alpha, IFNβ, IFNαs (e.g., INFα2b), GM-CSF, CD40L, Flt3 ligand, stem cell factor, ancestim, and TNFα. Suitable chemokines can include Glu-Leu-Arg (ELR)-negative chemokines such as IP-10, MCP-3, MIG, and SDF-1 alpha from the human CXC and C—C chemokine families. Suitable cytokines also include cytokine derivatives, cytokine variants, cytokine fragments, and cytokine fusion proteins

Immunomodulatory compounds may also include compounds that are agonists of a Toll-like receptor (TLR). “TLR” generally refers to any Toll-like receptor of any species of organism. Several human TLRs are disclosed in PCT publication no. WO 98/50547. Agonists of human TLRs are also described in Table 1 of Ulevich R, (2004) Nature Reviews: Immunology, 4:512-520; in Table 1 of Akira and Takeda (2004) Nature Reviews Immunology 4:499-511; in Medzhitov R, (2001) Nature Reviews Immunology 1:345-145; and in PCT publication nos. WO 03/031573 and WO 03/103586. Each of the preceding disclosures are incorporated herein by reference, including particularly the compounds listed in the references and Table 1 of the Ulevich (2004) and Akira and Takeda (2004) references.

A specific TLR may be identified with additional reference to species of origin (e.g., human, murine, etc.), a particular receptor (e.g., TLR3, TLR4, TLR6, TLR7, TLR8, TLR9, etc.), or both. “TLR agonist” refers to a compound that acts as an agonist of a TLR. Unless otherwise indicated, reference to a TLR agonist compound can include the compound in any pharmaceutically acceptable form, including any isomer (e.g., diastereomer or enantiomer), salt, solvate, polymorph, and the like. In particular, if a compound is optically active, reference to the compound can include each of the compound's enantiomers as well as racemic mixtures of the enantiomers. Also, a compound may be identified as an agonist of one or more particular TLRs (e.g., a TLR7 agonist, a TLR8 agonist, or a TLR7/8 agonist).

Certain TLRs are known to bind certain pathogen-associated ligands. In some cases the ligands are pathogen-derived, while in other cases the ligands are subjectderived. For example, TLR3 recognizes polyinosinic-polycytidylic acid (polyIC), a “mimic” of double-stranded viral RNA; TLR4 recognizes lipopolysaccharide (LPS) of many Gram-negative bacteria; TLR5 binds certain flagellins; and TLR9 binds certain CpG oligonucleotides. Certain small molecule IMC compounds are known to be agonists of one or more TLRs including, for example, TLR6, TLR7, and TLR8. In some embodiments, the TLR agonist may be an agonist of at least one of TLR6, TLR7, TLR8, and TLR9. In certain embodiment, the TLR agonist can be an agonist of TLR7 and/or TLR8. In alternative embodiments, the TLR agonist may be a TLR8-selective agonist. In other alternative embodiments, the TLR agonist can be a TLR7-selective agonist. As used herein, the term “TLR8-selective agonist” refers to any compound that acts as an agonist of TLR8, but does not act as an agonist of TLR7. A “TLR7-selective agonist” refers to a compound that acts as an agonist of TLR7, but does not act as an agonist of TLR8. A “TLR7/8 agonist” refers to a compound that acts as an agonist of both TLR7 and TLR8. A TLR8-selective agonist or a TLR7-selective agonist may act as an agonist for the indicated TLR and one or more of TLR1, TLR2, TLR3, TLR4, TLR5, TLR6, TLR9, or TLR10. Accordingly, while “TLR8-selective agonist” may refer to a compound that acts as an agonist for TLR8 and for no other TLR, it may alternatively refer to a compound that acts as an agonist of TLR8 and, for example, TLR6. Similarly, “TLR7-selective agonist” may refer to a compound that acts as an agonist for TLR7 and for no other TLR, but it may alternatively refer to a compound that acts as an agonist of TLR7 and, for example, TLR6. The TLR agonism for a particular compound may be assessed in any suitable manner. For example, assays for detecting TLR agonism of test compounds are described, for example, in PCT publication nos. WO 03/31573, WO 04/053057, WO 04/053452, and WO 04/094671 Regardless of the particular assay employed, a compound can be identified as an agonist of a particular TLR if performing the assay with a compound results in at least a threshold increase of some biological activity mediated by the particular TLR. Conversely, a compound may be identified as not acting as an agonist of a specified TLR if, when used to perform an assay designed to detect biological activity mediated by the specified TLR, the compound fails to elicit a threshold increase in the biological activity. Unless otherwise indicated, an increase in biological activity refers to an increase in the same biological activity over that observed in an appropriate control. An assay may or may not be performed in conjunction with the appropriate control. With experience, one skilled in the art may develop sufficient familiarity with a particular assay (e.g., the range of values observed in an appropriate control under specific assay conditions) that performing a control may not always be necessary to determine the TLR agonism of a compound in a particular assay. The precise threshold increase of TLR-mediated biological activity for determining whether a particular compound is or is not an agonist of a particular TLR in a given assay may vary according to factors known in the art including but not limited to the biological activity observed as the endpoint of the assay, the method used to measure or detect the endpoint of the assay, the signal-to-noise ratio of the assay, the precision of the assay, and whether the same assay is being used to determine the agonism of a compound for multiple TLRs.

In certain embodiments, the TLR agonist can be a natural agonist of a TLR or a synthetic IMC compound. WO 04/060319 lists a number of compounds suitable for use as IMCs, described as follows. IMC that may be useful as TLR agonists in immunostimulatory combinations of the invention are small organic molecules (e.g., molecular weight less than about 1000 Daltons, and less than about 500 Daltons in some cases), as opposed to large biological molecules such as proteins, peptides, and the like. Certain small molecule ICM compounds are disclosed in, for example, U.S. Pat. Nos. 4,689,338; 4,929,624; 4,988,815; 5,037,986; 5,175,296; 5,238,944; 5,266,575; 5,268,376; 5,346,905; 5,352,784; 5,367,076; 5,389,640; 5,395,937; 5,446,153; 5,482,936; 5,693,811; 5,741,908; 5,756,747; 5,939,090; 6,039,969; 6,083,505; 6,110,929; 6,194,425; 6,245,776; 6,331,539; 6,376,669; 6,451,810; 6,525,064; 6,545,016; 6,545,017; 6,558,951; and 6,573,273; European Patent 0 394 026; U.S. Patent Publication No. 2002/0055517; and International Patent Publication Nos.; WO 02/46188; WO 02/46189; WO 02/46190; WO 02/46191; WO 02/46192; WO 03/045391.

IMCs may also include purine derivatives (such as those described in U.S. Pat. Nos. 6,376,501, and 6,028,076), certain imidazoquinoline amide derivatives (such as those described in U.S. Pat. No. 6,069,149), certain benzimidazole derivatives (such as those described in U.S. Pat. No. 6,387,938), and certain derivatives of a 4-aminopyrimidine fused to a five membered nitrogen containing heterocyclic ring (such as adenine derivatives described in U.S. Pat. Nos. 6,376,501; 6,028,076 and 6,329,381; and in WO 02/085905). Other IMCs include large biological molecules such as oligonucleotide sequences. Some oligonucleotide sequences contain cytosine-guanine dinucleotides (CpG) and are described, for example, in U.S. Pat. Nos. 6,194,388; 6,207,646; 6,239,116; 6,339,068; and 6,406,705. Some CpG-containing oligonucleotides can include synthetic immunomodulatory structural motifs such as those described, for example, in U.S. Pat. Nos. 6,426,334 and 6,476,000. Other IMC nucleotide sequences lack CpG and are described, for example, in International Patent Publication No. WO 00/75304. CpG nucleic acids are known to be TLR9 agonists. As broadly defined, a CpG nucleic acid is a nucleic acid molecule, having at least one CpG dinucleotide motif in which at least the C of the dinucleotide is unmethylated. CpG nucleic acids include but are not limited to A class, B class and C class CpG nucleic acids. These classes of CpG nucleic acid have differing properties and activation profiles. Any other suitable CpG nucleic acid can be envisioned as well, generally where the nucleic acid molecule has an immunostimulatory property. The B class of CpG oligonucleotides are synthesized with nuclease resistant phosphorothioate backbones and are generally characterized by good B-cell and DC activation, but only limited NK cell activation. The A class of CpG oligonucleotides are synthesized with a chimeric backbone where the 5′ and 3′ ends are phosphorothioate and the central CpG motif region is phosphodiester. These oligonucleotides are characterized by good NK cell and DC activation leading to greater production of IFN-gamma but limited B-cell activation. The C class of CpG oligonucleotides are synthesized with a phosphorothioate backbone and have stimulatory properties intermediate to the other two classes of CpG oligonucleotides (e.g., good activation of B-cells as well as activation of NK cells and DCs). The methods of the invention preferably involve the use of A class, B class and C class CpG immunostimulatory nucleic acids. For B class CpG nucleic acids see, e.g., U.S. Pat. Nos. 6,194,388; 6,207,646; 6,214,806; 6,218,371; 6,239,116; and 6,339,068. For the A class CpG nucleic acids see, for example, published patent application PCT/US00/26527 (WO 01/22990). For C-class CpG nucleic acids, see U.S. provisional patent application 60/313,273, filed Aug. 17, 2001, U.S. Ser. No. 10/224,523 filed on Aug. 19, 2002, and US the entire contents of which are incorporated herein by reference.

Small molecule ICM compounds suitable for use as a TLR agonist in immunostimulatory combinations of the invention include compounds having a 2-aminopyridine fused to a five membered nitrogen-containing heterocyclic ring. Preferred example of such compounds are those which are TLR7 and/or TLR8 agonists. Such compounds include, for example, imidazoquinoline amines including but not limited to substituted imidazoquinoline amines such as, for example, aminoalkyl-substituted imidazoquinoline amines, amide-substituted imidazoquinoline amines, sulfonamide-substituted imidazoquinoline amines, urea-substituted imidazoquinoline amines, aryl ether-substituted imidazoquinoline amines, heterocyclic ether-substituted imidazoquinoline amines, amido ether-substituted in-Lidazoquinoline amines, sulfonamido ether-substituted imidazoquinoline amines, urea-substituted iniidazoquinoline ethers, and thioether-substituted imidazoquinoline amines; tetrahydroimidazoquinoline amines including but not limited to amide-substituted tetrahydroimidazoquinoline amines, sulfonamido-substituted tetrahydroimidazoquinoline amines, urea-substituted tetrahydroimidazoquinoline amines, aryl ether-substituted tetrahydroimidazoquinoline amines, heterocyclic ether-substituted tetrahydroimidazoquinoline hmines, amido ether-substituted tetrahydroimidazoquinoline amines, sulfonamido ether-substituted tetrahydroimidazoquinoline amines, urea-substituted tetrahydroimidazoquinoline ethers, and thioether-substituted tetrahydroimidazoquinoline amines; imidazopyridine amines including but not limited to amide-substituted imidazopyridine amines, sulfonamido-substituted imidazopyridine amines, urea-substituted imidazopyridine amines; aryl ether-substituted imidazopyridine amines, heterocyclic ether-substituted imidazopyridine amines, amido ether-substituted imidazopyridine amines, sulfonamido ether-substituted imidazopyridine amines, urea-substituted imidazopyridine ethers, and thioether-substituted imidazopyridine amines; 1,2-bridged imidazoquinoline amines; 6,7-fused cycloalkylimidazopyridine amines; imidazonaphthyridine amines; tetrahydroimidazonaphthyridine amines; oxazoloquinoline amines; thiazoloquinoline amines; oxazolopyridine amines; thiazolopyridine amines; oxazolonaphthyridine amines; and thiazolonaphthyridine amines.

Most preferably, the TLR agonist and IMC is imiquimod, whose chemical name is 1-(2-amino-2-methylpropyl)-2-(ethoxymethyl)-1H-imidazo[4,5-c]quinolin-4-amine or 4-Amino-1-isobutyl-1H-imidazo[4,5-c]quinoline. In certain embodiments, the TLR agonist may be an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, or a thiazolonaphthyridine amine. In certain embodiments, the TLR agonist can be a sulfonamide-substituted imidazoquinoline amine. In alternative embodiments, the TLR agonist can be a urea-substituted imidazoquinoline ether. In another alternative embodiment, the TLR agonist can be an aminoalkyl-substituted imidazoquinoline amine. In one particular embodiment, the TLR agonist is 4-amino-α,α,2-trimethyl-1H-imidazo[4,5-c]quinolin-1-ethanol. In an alternative particular embodiment, the TLR agonist is N-(2-{2-[4-amino-2-(2-methoxyethyl)-1H-imidazo[4,5-c]quinolin-1-yl]ethoxy}ethyl)-N-methylmorpholine-4-carboxamide. In another alternative embodiment, the TLR agonist is N-[4-(4-amino-2-ethyl-1H-imidazo[4,5-c]quinolin-1-yl)butyl}methanesulfonamide. In yet another alternative embodiment, the TLR agonist is N-[4-(4-amino-2propyl-1H-imidazo[4,5-c]quinolin-1-yl)butyl]methanesulfonamide.

In certain alternative embodiments, the TLR-agonist may be a substituted imidazoquinoline amine, a tetrahydroimidazoquinoline amine, an imidazopyridine amine, a 1,2-bridged imidazoquinoline amine, a 6,7-fused cycloalkylimidazopyridine amine, an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, or a thiazolonaphthyridine amine.

As used herein, a substituted imidazoquinoline amine refers to an aminoalkylsubstituted imidazoquinoline amine, an amide-substituted imidazoquinoline amine, a sulfonamide-substituted imidazoquinoline amine, a urea-substituted imidazoquinoline amine, an aryl ether-substituted imidazoquinoline amine, a heterocyclic ether-substituted imidazoquinoline amine, an amido ether-substituted imidazoquinoline amine, a sulfonamido ether-substituted imidazoquinoline amine, a urea-substituted imidazoquinoline ether, or a thioether-substituted imidazoquinoline amines. As used herein, substituted imidalzoquinoline amines specifically and expressly exclude 1-(2methylpropyl)-IH-imidazo[4,5-c]quinolin amine and 4-amino-a,a-dimethyl ethoxymethyl-IH-imidazo[4,5-c]quinolin-1-ethanol.

For example, the TLR agonist can be administered in an amount from about 100 ug/kg to about 100 mg/kg. In many embodiments, the TLR agonist is administered in an amount from about 10 ug/kg to about 10 mg/kg. In some embodiments, the TLR agonist is administered in an amount from about 1 mg/kg to about 5 mg/kg.

Immunogenic Compounds: Antigens

Preferred immunogenic compounds (IC) suitable for use according to the invention are antigens, particularly microbial—bacterial, viral and fungal—antigens and tumor or cancer antigens.

Tumor and cancer antigens are particularly well suited for intra-tumoral administration. A “cancer antigen” or “tumor antigen” as used herein is a compound, such as a peptide, associated with a tumor or cancer cell surface and which is capable of provoking an immune response when expressed on the surface of an antigen presenting cell in the context of an MHC molecule. Cancer antigens can be prepared from cancer cells either by preparing crude extracts of cancer cells, for example, as described in Cohen, et al., 1994, Cancer Research, 54:1055, by partially purifying the antigens, by recombinant technology, or by de novo synthesis of known antigens. Cancer antigens include antigens that are recombinately an immunogenic portion of or a whole tumor or cancer. Such antigens can be isolated or prepared recombinately or by any other means known in the art.

Tumor antigens can include tumour rejection antigens such as those for prostate, breast, colorectal, lung, pancreatic, renal or melanoma cancers. Exemplary antigens include MAGE 1 and MAGE 3 or other MAGE antigens (for the treatment of melanoma), PRAME, BAGE, or GAGE (Robbins and Kawakami, 1996, Current Opinions in Immunology 8, pps 628-636; Van den Eynde et al., International Journal of Clinical & Laboratory Research (submitted 1997); Correale et al. (1997), Journal of the National Cancer Institute 89, p 293. Indeed these antigens are expressed in a wide range of tumour types such as melanoma, lung carcinoma, sarcoma and bladder carcinoma. Other tumour-specific antigens are suitable for use with the adjuvants of the present invention and include, but are not restricted to tumour-specific gangliosides, Prostate specific antigen (PSA) or Her-2/neu, KSA (GA733), PAP, mammaglobin, MUC-1, carcinoembryonic antigen (CEA). Accordingly in one aspect of the present invention there is provided a vaccine comprising an adjuvant composition according to the invention and a tumour rejection antigen.

A “microbial antigen” as used herein is an antigen of a microorganism and includes but is not limited to infectious virus, infectious bacteria, infectious parasites and infectious fungi. Such antigens include the intact microorganism as well as natural isolates and fragments or derivatives thereof and also synthetic compounds which are identical to or similar to natural microorganism antigens and induce an immune response specific for that microorganism. A compound is similar to a natural microorganism antigen if it induces an immune response (humoral and/or cellular) to a natural microorganism antigen. Most such antigens are used routinely in the art and are well known to those of ordinary skill in the art. Another example is a peptide mimic of a polysaccharide antigen.

Antigens may be derived from infectious virus of both human and non-human vertebrates, include retroviruses, RNA viruses and DNA viruses. This group of retroviruses includes both simple retroviruses and complex retroviruses. The simple retroviruses include the subgroups of B-type retroviruses, C-type retroviruses and D-type retroviruses. An example of a B-type retrovirus is mouse mammary tumor virus (MMTV). The C-type retroviruses include subgroups C-type group A (including Rous sarcoma virus (RSV), avian leukemia virus (ALV), and avian myeloblastosis virus (AMV)) and C-type group B (including murine leukemia virus (MLV), feline leukemia virus (FeLV), murine sarcoma virus (MSV), gibbon ape leukemia virus (GALV), spleen necrosis virus (SNV), reticuloendotheliosis virus (RV) and simian sarcoma virus (SSV)). The D-type retroviruses include Mason-Pfizer monkey virus (MPMV) and simian retrovirus type 1 (SRV-1). The complex retroviruses include the subgroups of lentiviruses, T-cell leukemia viruses and the foamy viruses. Lentiviruses include HIV-1, but also include HIV-2, SIV, Visna virus, feline immunodeficiency virus (FIV), and equine infectious anemia virus (EIAV). The T-cell leukemia viruses include HTLV-1, HTLV-II, simian T-cell leukemia virus (STLV), and bovine leukemia virus (BLV). The foamy viruses include human foamy virus (HFV), simian foamy virus (SFV) and bovine foamy virus (BFV).

Examples of other RNA viruses that are antigens in mammals include, but are not limited to, the following: members of the family Reoviridae, including the genus Orthoreovirus (multiple serotypes of both mammalian and avian retroviruses), the genus Orbivirus (Bluetongue virus, Eugenangee virus, Kemerovo virus, African horse sickness virus, and Colorado Tick Fever virus), the genus Rotavirus (human rotavirus, Nebraska calf diarrhea virus, murine rotavirus, simian rotavirus, bovine or ovine rotavirus, avian rotavirus); the family Picornaviridae, including the genus Enterovirus (poliovirus, Coxsackie virus A and B, enteric cytopathic human orphan (ECHO) viruses, hepatitis A virus, Simian enteroviruses, Murine encephalomyelitis (ME) viruses, Poliovirus muris, Bovine enteroviruses, Porcine enteroviruses, the genus Cardiovirus (Encephalomyocarditis virus (EMC), Mengovirus), the genus Rhinovirus (Human rhinoviruses including at least 113 subtypes; other rhinoviruses), the genus Apthovirus (Foot and Mouth disease (FMDV); the family Calciviridae, including Vesicular exanthema of swine virus, San Miguel sea lion virus, Feline picornavirus and Norwalk virus; the family Togaviridae, including the genus Alphavirus (Eastern equine encephalitis virus, Semliki forest virus, Sindbis virus, Chikungunya virus, O'Nyong-Nyong virus, Ross river virus, Venezuelan equine encephalitis virus, Western equine encephalitis virus), the genus Flavirius (Mosquito borne yellow fever virus, Dengue virus, Japanese encephalitis virus, St. Louis encephalitis virus, Murray Valley encephalitis virus, West Nile virus, Kunjin virus, Central European tick borne virus, Far Eastern tick borne virus, Kyasanur forest virus, Louping III virus, Powassan virus, Omsk hemorrhagic fever virus), the genus Rubivirus (Rubella virus), the genus Pestivirus (Mucosal disease virus, Hog cholera virus, Border disease virus); the family Bunyaviridae, including the genus Bunyvirus (Bunyamwera and related viruses, California encephalitis group viruses), the genus Phlebovirus (Sandfly fever Sicilian virus, Rift Valley fever virus), the genus Nairovirus (Crimean-Congo hemorrhagic fever virus, Nairobi sheep disease virus), and the genus Uukuvirus (Uukuniemi and related viruses); the family Orthomyxoviridae, including the genus Influenza virus (Influenza virus type A, many human subtypes); Swine influenza virus, and Avian and Equine Influenza viruses; influenza type B (many human subtypes), and influenza type C (possible separate genus); the family paramyxoviridae, including the genus Paramyxovirus (Parainfluenza virus type 1, Sendai virus, Hemadsorption virus, Parainfluenza viruses types 2 to 5, Newcastle Disease Virus, Mumps virus), the genus Morbillivirus (Measles virus, subacute sclerosing panencephalitis virus, distemper virus, Rinderpest virus), the genus Pneumovirus (respiratory syncytial virus (RSV), Bovine respiratory syncytial virus and Pneumonia virus of mice); forest virus, Sindbis virus, Chikungunya virus, O'Nyong-Nyong virus, Ross river virus, Venezuelan equine encephalitis virus, Western equine encephalitis virus), the genus Flavirius (Mosquito borne yellow fever virus, Dengue virus, Japanese encephalitis virus, St. Louis encephalitis virus, Murray Valley encephalitis virus, West Nile virus, Kunjin virus, Central European tick borne virus, Far Eastern tick borne virus, Kyasanur forest virus, Louping III virus, Powassan virus, Omsk hemorrhagic fever virus), the genus Rubivirus (Rubella virus), the genus Pestivirus (Mucosal disease virus, Hog cholera virus, Border disease virus); the family Bunyaviridae, including the genus Bunyvirus (Bunyamwera and related viruses, California encephalitis group viruses), the genus Phlebovirus (Sandfly fever Sicilian virus, Rift Valley fever virus), the genus Nairovirus (Crimean-Congo hemorrhagic fever virus, Nairobi sheep disease virus), and the genus Uukuvirus (Uukuniemi and related viruses); the family Orthomyxoviridae, including the genus Influenza virus (Influenza virus type A, many human subtypes); Swine influenza virus, and Avian and Equine Influenza viruses; influenza type B (many human subtypes), and influenza type C (possible separate genus); the family paramyxoviridae, including the genus Paramyxovirus (Parainfluenza virus type 1, Sendai virus, Hemadsorption virus, Parainfluenza viruses types 2 to 5, Newcastle Disease Virus, Mumps virus), the genus Morbillivirus (Measles virus, subacute sclerosing panencephalitis virus, distemper virus, Rinderpest virus), the genus Pneumovirus (respiratory syncytial virus (RSV), Bovine respiratory syncytial virus and Pneumonia virus of mice); the family Rhabdoviridae, including the genus Vesiculovirus (VSV), Chandipura virus, Flanders-Hart Park virus), the genus Lyssavirus (Rabies virus), fish Rhabdoviruses, and two probable Rhabdoviruses (Marburg virus and Ebola virus); the family Arenaviridae, including Lymphocytic choriomeningitis virus (LCM), Tacaribe virus complex, and Lassa virus; the family Coronoaviridae, including Infectious Bronchitis Virus (IBV), Mouse Hepatitis virus, Human enteric corona virus, and Feline infectious peritonitis (Feline coronavirus).

Illustrative DNA viruses that are antigens in mammals include, but are not limited to: the family Poxviridae, including the genus Orthopoxvirus (Variola major, Variola minor, Monkey pox Vaccinia, Cowpox, Buffalopox, Rabbitpox, Ectromelia), the genus Leporipoxvirus (Myxoma, Fibroma), the genus Avipoxvirus (Fowlpox, other avian poxvirus), the genus Capripoxvirus (sheeppox, goatpox), the genus Suipoxvirus (Swinepox), the genus Parapoxvirus (contagious postular dermatitis virus, pseudocowpox, bovine papular stomatitis virus); the family Iridoviridae (African swine fever virus, Frog viruses 2 and 3, Lymphocystis virus of fish); the family Herpesviridae, including the alpha-Herpesviruses (Herpes Siruplex Types 1 and 2, Varicella-Zoster, Equine abortion virus, Equine herpes virus 2 and 3, pseudorabies virus, infectious bovine keratoconjunctivitis virus, infectious bovine rhinotracheitis virus, feline rhinotracheitis virus, infectious laryngotracheitis virus) the Beta-herpesvirises (Human cytomegalovirus and cytomegaloviruses of swine, monkeys and rodents); the gamma-herpesviruses (Epstein-Barr virus (EBV), Marek's disease virus, Herpes saimiri, Herpesvirus ateles, Herpesvirus sylvilagus, guinea pig herpes virus, Lucke tumor virus); the family Adenoviridae, including the genus Mastadenovirus (Human subgroups A,B,C,D,E and ungrouped; simian adenoviruses (at least 23 serotypes), infectious canine hepatitis, and adenoviruses of cattle, pigs, sheep, frogs and many other species, the genus Aviadenovirus (Avian adenoviruses); and non-cultivatable adenoviruses; the family Papoviridae, including the genus Papillomavirus (Human papilloma viruses, bovine papilloma viruses, Shope rabbit papilloma virus, and various pathogenic papilloma viruses of other species), the genus Polyomavirus (polyomavirus, Simian vacuolating agent (SV-40), Rabbit vacuolating agent (RKV), K virus, BK virus, JC virus, and other primate polyoma viruses such as Lymphotrophic papilloma virus); the family Parvoviridae including the genus Adeno-associated viruses, the genus Parvovirus (Feline panleukopenia virus, bovine parvovirus, canine parvovirus, Aleutian mink disease virus, etc). Finally, DNA viruses may include viruses which do not fit into the above families such as Kuru and Creutzfeldt-Jacob disease viruses and chronic infectious neuropathic agents (CHINA virus).

Other preferred exemplary antigens are HPV antigens from any strain of HPV. HPV expresses six or seven non-structural and two structural proteins. Viral capsid proteins L1 and L2 are the late structural proteins. L1 is the major capsid protein, the amino acid sequence of which is highly conserved among different HPV types. There are seven early non-structural proteins. Proteins E1, E2, and E4 play an important role in virus replication. Protein E4 also plays a role in virus maturation. The role of E5 is less well known. Proteins E6 and E7 are oncoproteins critical for viral replication, as well as for host cell immortalization and transformation. Fusion proteins of the invention can contain either the entire sequence of an HPV protein or a fragment thereof, e.g., a fragment of at least 8 amino acids. In one embodiment, the HPV antigenic sequence is derived from a “high risk” HPV, such as HPV16 or HPV18 E7 protein. The HPV antigenic sequence can include an MHC-binding epitope, e.g., an MHC class I and/or an MHC class II binding epitope.

Other antigens may be derived from bacteria, parasites or yeast. Examples of suitable species include Neisseria spp, including N. gonorrhea and N. meningitidis (for example, capsular polysaccharides and conjugates thereof, transferrin-binding proteins, lactoferrin binding proteins, PilC and adhesions can be used as antigens); S. pyogenes (for example M proteins or fragments thereof, C5A protease, lipoteichoic acids), S. agalactiae, S. mutans; H. ducreyi; Moraxella spp, including M. catarrhalis, also known as Branhamella catarrhalis (for example high and low molecular weight adhesins and invasins); Bordetella spp, including B. pertussis (for example pertactin, pertussis toxin or derivatives thereof, filamenteous hemagglutinin, adenylate cyclase, fimbriae), B. parapertussis and B. bronchiseptica; Mycobacterium spp., including M. tuberculosis (for example ESAT6, Antigen 85A, -B or -Q, M. bovis, M leprae, M avium, M. paratuberculosis, M. smegmatis; Legionella spp, including L. pneumophila; Escherichia spp, including enterotoxic E. coli (for example colonization factors, heat-labile toxin or derivatives thereof, heat-stable toxin or derivatives thereof), enterohemorragic E. coli, enteropathogenic E. coli (for example io shiga toxin-like toxin or derivatives thereof); Vibrio spp, including V. cholera (for example cholera toxin or derivatives thereop; Shigella spp, including S. sonnei, S. dysenteriae, S. flexnerii; Yersinia spp, including Y enterocolitica (for example a Yop protein), Y. pestis, Y. pseudotuberculosis; Campylobacter spp, including C jejuni (for example toxins, adhesins and invasins) and C coli; Salmonella spp, including S. typhip S. paratyphi, S. choleraesuis, S. enteritidis; Listeria spp., including L. monocytogenes; Helicobacter spp, including H. pylori (for example urease, catalase, vacuolating toxin); Pseudomonas spp, including P. aeruginosa; Staphylococcus spp., including S. aureus, S. epidermidis; Enterococcus spp., including E. jaecalis, E. jaecium; Clostridium spp., including C tetani (for example tetanus toxin and derivatives thereof), C botulinum (for example botulinum toxin and derivatives thereof, C difficile (for example clostridium toxins A or B and derivatives thereof); Bacillus spp., including B. anthracis (for example botulinum toxin and derivatives thereof); Corynebacterium spp., including C diphtheriae (for example diphtheria toxin and derivatives thereof); Borrelia spp., including B. burgdorferi (for example OspA, OspC, DbpA, DbpB), B. garinii (for example OspA, OspC, DbpA, DbpB), B. afzelii (for example OspA, OspC, DbpA, DbpB), B. andersonii (for example OspA, OspC, DbpA, DbpB), B. hermsii; Ehrlichia spp., including E. equi and the agent of the Human Granulocytic Ehrlichiosis; Rickettsia spp, including R. rickettsii; Chlamydia spp., including C trachomatis (for example MOMP, heparin-binding proteins), C. pneumoniae (for example MONT, heparin-binding proteins), C psittaci; Leptospira spp., including L. interrogans; Treponema spp., including T. pallidum (for example the rare outer membrane proteins), T denticola, T hyodysenteriae; or species derived from parasites such as Plasmodium spp., including P. falciparum; Toxoplasma spp., including T. gondii (for example SAG2, SAG3, Yg34); Entamoeba spp., including E. histolytica; Babesia spp., including B. microti; Trypanosoma spp., including T cruzi; Giardia spp., including G. lamblia; Leshmania spp., including L. major; Pneumocystis spp., including P. carinii; Trichomonas spp., including T. vaginalis; Schisostoma spp., including S. mansoni, or species derived from yeast such as Candida spp., including C albicans; Cryptococcus spp., including C neoformans.

Each of the foregoing lists is illustrative, and is not intended to be limiting. The disclosures of each of foregoing references disclosing antigens and diseases or conditions are incorporated herein by reference.

Mycobacterium Antigens

The Mycobacterial antigen for use according to the invention can be for example, and without to be limited thereto, live, killed or attenuated mycobacterium compositions, mycobacterial culture supernatants, mycobacterial cell extracts, cell wall fractions or cell wall elements, preferably purified cell wall elements, DNA fractions or purified DNA molecules, or mycobacterial peptide or non-peptidic (for example non-peptidic phosphorylated antigens) antigens, in the form of fractions enriched in said antigen or purified antigen. In addition to using attenuated or killed mycobacterial strains, it is also possible to use any of a variety of non-pathogenic or non-human infecting strains of Mycobacterium, such as for example M. phlei, M. piscium or M. smegmatis in naturally existing forms. Preferred mycobacterial antigens are compositions capable of regulating, preferably stimulating γδT cell activity.

In a preferred embodiment, said Mycobacterium antigen is an antigen of any one of Mycobacterium strains, more particularly a Mycobacterium strain selected from the group consisting of Mycobacterium avium, Mycobacterium bovis, Mycobacterium phlei, Mycobacterium tuberculosis, Mycobacterium paratuberculosis, Mycobacterium heamophilum, Mycobacterium leprae, Mycobacterium chelonae, Mycobacterium fortuitum, Mycobacterium kansasii, Mycobacterium marinum, Mycobacterium scrofulaceum, Mycobacterium smegmatis, Mycobacterium ulcerans, and Mycobacterium xenopi. More preferably, said Mycobacterium antigen is an antigen of a Mycobacterium strain selected from the group consisting of Mycobacterium bovis, Mycobacterium phlei, Mycobacterium tuberculosis, and Mycobacterium paratuberculosis. In a most preferred embodiment, said Mycobacterium antigen is an antigen of Mycobacterium bovis. In an other preferred embodiment, said Mycobacterium antigen is an antigen of Mycobacterium phlei, Mycobacterium vaccae or Mycobacterium piscium.

Preferably, said antigen of Mycobacterium is an attenuated strain thereof. Methods to prepare an attenuated strain are well-known by the man skilled in the art. For example, one method is disclosed in U.S. Pat. No. 6,403,100. The Mycobacterium antigen can be for example any attenuated strain of Mycobacterium bovis (Bacillus Calmette-Guérin—BCG). Mycobacterium bovis strain can be a BCG strain of ATCC number selected from the group consisting of: 19015; 19274; 27289; 27290; 27291; 35731; 35732; 35733; 35734; 35735; 35736; 35737; 35738; 35739; 35740; 35741; 35742; 35743; 35744; 3574. More particularly, it can be derived, without to be limited thereto, from the strains of Montreal or Pasteur Institute. For example, BCG can be PACIS BCG or TICE BCG. PACIS BCG is derived from Montreal strain which originates from a BCG culture given to Dr. Armand Frappier by Dr. C. Guérin in 1937. This strain was maintained by passaging until 1973. PACIS has also been known as “the Armand-Frappier strain of BCG”. The TICE strain was developed at the University of Illinois from a strain originated at the Pasteur Institute. This strain was maintained by continuous passage for more than 20 years. For example, BCG can be prepared by the following process. BCG is grown on glycerinized potato medium followed by further passages on Sauton medium. After harvesting by filtration, BCG are resuspended in a 15% (w/v) lactose solution, filled into container, and preferably lyophilized.

Further mycobacterial antigen compositions that can use in accordance with the invention may comprise a major mycobacterial antigen or a recombinant mycobacterial strains and DNA forms. For example, of major antigens that are targets of the immune response to infection by Mycobacteria have been reported in Kaufman, Immunol. Today 11: 129-136 (1990); Young, Ann. Rev. Immunol. 8: 401-420 (1990); Young et al., Academic Press Ltd., London, pp. 1-35, 1990; Young et al, Mol. Microbiol. 6: 133-145 (1992)]. Recombinant BCG vaccine vehicles have been proposed (Snapper et al., PNAS. USA. 85: 6987-6991 (1988); Husson et al., J. Bacteriol. 172: 519-524 (1990); Martin et al., Nature 345: 739-743 (1990); Snapper et al, Mol. Microbiol. 4: 1911-1919 (1990); Aldovini and Young, Nature 351: 479-482 (1991); Jacobs et al, Methods Enzymology 204:537-555 (1991); Lee et al, PNAS 88: 3111-3115 (1991); Stover et al., Nature 351:456-460 (1991); Winter et al., Gene 109: 47-54 (1991); Donnelly-Wu et al., Mol. Microbiol. 7: 407-417 (1993)). Examples of recombinant DNA forms and strains of Mycobacteria are provided in U.S. Pat. No. 5,866,403 describing the production and uses of homologously recombinant slow growing Mycobacteria; U.S. Pat. No. 5,854,055 describing recombinant Mycobacteria vaccine vehicles capable of expressing a foreign DNA of interest; U.S. Pat. No. 5,840,855 describing Mycobacterial recombinants and peptides encoded by the genome of Mycobacterium tuberculosis for use as vectors and protein expression; U.S. Pat. No. 5,830,475 presenting recombinant Mycobacterial vaccines which express a heterologous DNA encoding a protein or polypeptide product such as a cytokine; U.S. Pat. No. 5,807,723 offering a homologously recombinant slow growing Mycobacteria and methods of manipulating the genomic, DNA of slow growing Mycobacterial species; U.S. Pat. No. 5,504,005 describing a recombinant Mycobacterial vaccine capable of expressing a foreign DNA of interest against which an immune response is desired; U.S. Pat. No. 5,591,632 presenting a recombinant BCG-Mycobacteria expressing heterologous DNA encoding a polypeptide or protein for initiating an immune response; and U.S. Pat. No. 5,776,465 describing recombinant Mycobacterial vaccines, particularly a recombinant M. bovis BCG species which expresses heterologous DNA. Each of the references listed in this paragraph are incorporated herein by reference.

For the treatment of bladder cancer, the pharmaceutical composition comprising the Mycobacterium antigen, and optionally the γδT cell activator, can be administered locally at the disease site (the bladder) via intravesical treatment as following. The subject should not drink fluids for 4 hours before treatment and should empty its bladder prior administration of the pharmaceutical composition. The pharmaceutical composition is instilled into the bladder slowly (e.g., by gravity flow) via a catheter. The pharmaceutical composition is retained in the bladder for about two hours and then voided. During the period of treatment, more particularly during the first hour after the instillation, the subject should lie for 15 minutes each in the prone and suspine positions and also on each side, to maximize surface exposure to the pharmaceutical composition. Preferably, the treatment cycle consists of one intravesicular instillation per week for six weeks. Thereafter, the treatment can be continued at monthly intervals for 6-12 months. Optionally, the treated subjects can be evaluated, for example at 3, 6, and/or 9 months, after the treatment. The evaluation can be performed by cytoscopy, cytology and/or biopsy. Patients may also continue to be treated as maintenance therapy.

A preferred composition comprising a mycobacterial antigen for use in accordance with the invention is Immucyst® (Bacillus Calmette-Guérin (BCG), substrain Connaught) available from Aventis Pasteur. As reproduced from the ImmuCyst® drug label, the product is made from a culture of an attenuated strain of living bovine tubercle bacillus Mycobacterium bovis. The bacilli are lyophilized (freeze-dried) and are viable upon reconstitution. When plated on culture media, the progenitor of each colony is termed a “colony-forming unit” (CFU); each CFU is composed of at least one viable bacillus and may comprise several bacilli, some of which may be viable and some non-viable. Each vial contains 81 mg (dry weight) of BCG and 5% w/v monosodium glutamate. Each vial of ImmuCyst® is reconstituted with the accompanying diluent (3.0 mL), which consists of approximately 0.85% w/v sodium chloride, 0.025% w/v Tween 80, 0.06% w/v sodium dihydrogen phosphate and 0.25% w/v disodium hydrogen phosphate. The product and the diluent contain no preservative. One dose consists of one 81 mg vial of reconstituted material further diluted in 50 mL sterile, preservative-free saline. The reconstituted dose contains 10.5±8.7×10⁸ colony forming units (CFU) over the course of its shelf-life.

When administered intravesically as a cancer therapy, BCG promotes a local acute inflammatory and sub-acute granulomatous reaction with histiocytic and leukocytic infiltration in the urothelium and lamina propria of the urinary bladder. The local inflammatory effects are associated with an elimination or reduction of superficial cancerous lesions of the urinary bladder.

ImmuCyst® is commercialised for treatment of superficial transitional cell carcinoma (TCC) of the urinary bladder, including carcinoma in situ (CIS), papillary tumors limited to the mucosa (stage Ta), papillary tumors involving the lamina propria but not the muscle layer of the bladder (stage T1), or any combination thereof. ImmuCyst® is indicated for the treatment and prophylaxis of primary or recurrent carcinoma in-situ (CIS) of the urinary bladder, and for prophylaxis following TUR of primary or recurrent stage Ta and/or T1 papillary tumors.

ImmuCyst® is preferably dosed and administered according to the manufacturer's instructions as follows. Intravesical treatment of the urinary bladder using ImmuCyst® is recommended to begin between 7 to 14 days after biopsy or transurethral resection. The induction treatment comprises 6 weekly intravesical treatments with ImmuCyst®, each treatment dose comprising one 81 mg vial of ImmuCyst®. After a 6-week pause, another dose should be given intravesically once weekly for 1-3 weeks. Three weekly doses should definitely be given to patients who still have evidence of bladder cancer. Clinical studies have demonstrated that the 3 doses given at 3 months significantly increased the complete response rate from 73% to 87% at 6 months. Maintenance therapy following induction is recommended. This consists of 1-3 weekly treatments at 6 months following the initiation of treatment, and then every 6 months thereafter until 36 months.

Each dose (1 reconstituted vial) is further diluted in an additional 50 mL of sterile, preservative-free saline for a total of 53 mL (see reconstitution instructions below). A urethral catheter is inserted into the bladder under aseptic conditions, the bladder is drained, and then 53 mL suspension of ImmuCyst® is instilled slowly by gravity, following which the catheter is withdrawn. The patient retains the suspension for as long as possible for a total of up to two hours. During the first 15 minutes following instillation, the patient should lie prone. Thereafter, the patient is then allowed to be up. At the end of 2 hours, all patients should void in a seated position for environmental safety reasons. Patients should be instructed to maintain adequate hydration.

Another preferred mycobacterial antigen-containing composition made from a culture of an attenuated strain of living BCG is TICE™ BCG (Organon Teknika Corp.). TICE™ is commercialised for the treatment of carcinoma in situ of the bladder. TICE™ can be administered according to the manufacturer's instructions as follows, recommending that the product is administered 7-14 days after bladder biopsy. The reconstituted TICE™ is installed into the bladder by gravity flow using a catheter, is maintained in the bladder for two hours and then voided. While the BCG is in the bladder, the patient should be repositioned from the left side to the right side and the back side to the abdomen every 15 minutes in order to maximize surface exposure to the agent. A standard treatment of TICE consists of one intravesicular instillation per week for six weeks. The schedule may be repeated once if tumor remission has not been achieved. Thereafter, TICE™ administration can be continued at monthly intervals for 6-12 months.

Another preferred mycobacterial antigen-containing composition made from a culture of an attenuated strain of living BCG is Pacis™ (Shire Biologics, Sainte-Foy, Quebec, Canada and Urocor, Inc., Oklahoma USA). Pacis™ is supplied as a single dose ampule of 120 mg (semi-dry weight) lyophilised BCG (2.4 to 12×10⁸ C.F.U. per ampule). Pacis™ is commercialised for the treatment of bladder cancer and treatment. Pacis™ can be administered in accordance with the manufacturer's instructions at a single dose of 120 mg according to the same methods as TICE™ once weekly for six-weeks, which cycle may be repeated if tumor remission has not been achieved.

Another preferred mycobacterial antigen-containing composition is BCG-Medac, made from a culture of an attenuated strain of living BCG, strain RIVM derived from strain 1173-P2 (medac, Hamburg, Germany). BCG-medac is commercialised for the treatment of bladder cancer and treatment can be carried out in accordance with the manufacturer's instructions as follows. The reconstituted dose of BCG-medac contains 2×10⁸−3×10⁰ colony forming units (CFU) of attenuated BCG and is installed into the bladder 2 to 3 weeks after transurethral resection using a single use catheter under slight pressure. A standard treatment of BCG-medac consists of one intravesicular instillation per week for six weeks. The schedule may be repeated if tumor remission has not been achieved. After a treatment-free period of 4 weeks, BCG-medac administration can be continued at monthly intervals for a 12 month period. Alternatively, BCG-medac can be given for six weeks followed by weekly injections for three consecutive weeks in months 3, 6, 12, 18, 24, 30 and 36.

An efficient dose of an attenuated BCG strain according the present invention preferably comprises about 0.1 to 50×10⁸ colony forming units, more preferably 1 to 15 10⁸ colony forming units.

Yet another preferred mycobacterial antigen-containing composition is the product referred to as SRL172 (SR Pharma, London, U.K.), a killed Mycobacterium vaccae suspension also described in PCT application no. WO85/03639, the disclosure of which is incorporated herein by reference.

Preparations of bacterial origin, including, but not limited to, preparations from Mycobacterium species, have been used to treat cancers (U.S. Pat. No. 4,503,048, the disclosure of which is incorporated herein by reference). One example is REGRESSIN™, a non-viable mycobacterial cell wall extract (MCWE) formulated as a mineral oil emulsion (Bioniche, Inc. London, Ontario, Canada), which has been shown to reduce cancer burden in bladder cancers (Kadhim et al. Journal of Urology 149:A255, 1996; Morales et al. Journal of Urology 157:A214, 1997). MCWE is composed primarily of peptidoglycan and glycolipid (Chin et al. Journal of Urology 156:1189-1193, 1996) and contain N-acetylmuramyl-L-alanyl-D-isoglutamine (muramyl dipeptide) and mycolic acid derivatives. Both muramyl dipeptide and mycolic acid derivatives stimulate the immune system by activation of macrophage and monocyte mediated reactions (Mallick et al. Comparative Immunology and Microbiology of Infectious Diseases 8:55-63, 1985; Teware et al. Veterinary Parasitology 62:223-230, 1996).

Another preparation that can be used in accordance with the invention is a Mycobacterium cell wall composition, preferably deproteinized and delipidated and optionally complexed to DNA. Such a composition comprising a Mycobacterium phlei deoxyribonucleic acid (M-DNA)-Mycobacterium phlei cell wall complex (MCC) is provided in U.S. Pat. No. 6,329,347, the disclosure of which is incorporated herein by reference.

Another preferred preparation is a DNA-rich fraction extracted and purified from mycobacterium bovis BCG referred to as MY-1. MY-1 is described in Fujeida et al, (1999) Am. J. Respir. Crit. Care med. 160: 2056-2061, and the preparation of MY-1 is described in Tokunaga et al, (1984) J. Natl. Cancer Inst. 72:955-962, both of which disclosures are incorporated herein by reference.

γδT Cell Activators

The term “γδT cell activator” designates a molecule, preferably artificially produced, which can activate γδT lymphocytes. It is more preferably a ligand of the T receptor of γδT lymphocytes. The activator may by of various nature, such as a peptide, lipid, small molecule, etc. It may be a purified or otherwise artificially produced (e.g., by chemical synthesis, or by microbiological process) endogenous ligand, or a fragment or derivative thereof, or an antibody having substantially the same antigenic specificity.

The γδ T cell activator preferably increases the biological activity or causes the proliferation of γδ T cells, preferably increasing the activation of γδ T cells, particularly increasing cytokine secretion from γδ T cells or increasing the cytolytic activity of γδ T cells, with or without also stimulating the proliferation or expansion of γδ T cells. Accordingly, the γδ T cell activator is administered in an amount and under conditions sufficient to increase the activity γδ T cells in a subject, preferably in an amount and under conditions sufficient to increase cytokine secretion by γδ T cells and/or to increase the cytolytic activity of γδ T cells. Cytokine secretion and cytolytic activity as well as γδ T cell proliferation can be assessed using any appropriate in vitro assay.

Most preferably the γδ T cells referred to in the present specification are Vγ9Vδ2 T cells, and preferably the γδ T cell activator is a Vγ9Vδ2 T cell activator.

In one example, γδ T cell activation can be assessed by administering a compound to an individual (human or non-human primate) and assessing activation or proliferation of Vγ9Vδ2 T cell. In an exemplary protocol expansion of the Vγ9Vδ2 T cell population is assessed: a candidate γδ T lymphocyte activator is administered to a non-human primate such as a cynomolgus monkey by intravenous infusion (one administration by slow infusion, 50 ml over 30 minutes) in combination with IL-2 (0.9 million units twice daily by subcutaneous injection for 5 days); peripheral γδ lymphocytes are analysed by flow cytometry on total monkey blood, after double staining with anti-CD3-PE antibody and anti-Vgamma9-FITC antibodies and/or anti Vd2 antibodies, and cells are counted by flow cytometry. Peak expansion of the Vγ9Vδ2 T cell population is observed between days 3 and 8, generally at about days 4-6 after administration of the γδ T lymphocyte activator.

Any other suitable tests can be used to assess cell proliferation. Assessment of proliferation or peripheral γδ lymphocytes can generally be analyzed by flow cytometry on total blood (for example total blood obtained from a monkey), after double staining with anti-CD3-PE antibody and anti-Vgamma9-FITC antibodies and/or anti Vd2 antibodies (CD3-PE: SP34 clone, BD Biosciences Pharmingen, Le Pont de Claix, France). Anti Vgamma 9, clone 7B6 is a monoclonal raised to human Vgamma 9 but that cross-reacts with cynomolgus monkey cells. It is purified by affinity chromatography on protein A and coupled to FITC. 50 μl monkey blood is incubated 15 min at RT with 5 μl anti-CD3-PE and 6 μl anti-delta2-FITC or 10 μl anti-gamma9-FITC antibodies. Antibodies are washed with 3 ml 1×PBS, centrifuged for 4 min at 1300 rpm at RT and supernatant is discarded. Red cells are lysed with the OptiLyse C reagent (Immunotech-Beckman-Coulter, Marseilles, France) according to the manufacturer's instructions. At the final step, stained white blood cells are recovered by centrifugation and resuspended in 300 μl PBS+0.2% PFA. Immediately before analysis, 50 μl calibrated Flow Count™ Fluorospheres (Immunotech-Beckman-Coulter, Marseilles, France) are added to the cells for absolute number counting of the populations of interest.

Preferably a γδ T lymphocyte activator is a compound capable of regulating the activity of a γδ T cell in a population of γ [symbol] T cell clones in culture. The γδ T lymphocyte activator is more preferably capable of regulating the activity of a γ [symbol] T cell population of γ [symbol] T cell clones in a at millimolar concentration, preferably when the γ [symbol] T cell activator is present in culture at a concentration of less than 100 mM. In one example, cytokine production or release is assessed. Vg9Vd2 cells are known producers of TNFα and IFNγ in vitro upon administration of the γ [symbol] T cell activator. Shortly after γ [symbol] T cell activator treatment, samples of sera are collected from an individual and are assayed by ELISA specific for TNFα or IFNγ.

Regulating the activity of a γ [symbol] T cell can be assessed by any suitable means, preferably by assessing cytokine secretion, most preferably TNF-α secretion as described herein. Methods for obtaining a population of pure γ [symbol] T cell clones is described in Davodeau et al, ((1993) J. Immunology 151(3): 1214-1223) and Moreau et al, ((1986) J. Clin. Invest. 78:874), the disclosures of which are incorporated herein by reference.

In any exemplary assay, cytokine secretion can be determined according to the methods described in Espinosa et al. (J. Biol. Chem., 2001, Vol. 276, Issue 21, 18337-18344), describing measurement of TNF-α release in a bioassay using TNF-α-sensitive cells. Briefly, 10⁴ γδT cells/well were incubated with stimulus plus 25 units of IL2/well in 100 μl of culture medium during 24 h at 37° C. Then, 50 μl of supernatant were added to 50 μl of WEHI cells plated at 3×10⁴ cells/well in culture medium plus actinomycin D (2 μg/ml) and LiCl (40 mM) and incubated for 20 h at 37° C. Viability of the TNF-α-sensitive cells and measured with a 3-(4,5-dimethylthiazol-2-yl)-2,5-diphenyltetrazolium bromide assay. 50 μl of 3-(4,5-dimethylthiazol-2-yl)-2,5-diphenyltetrazolium bromide (Sigma; 2.5 mg/ml in phosphate-buffered saline) per well were added, and after 4 h of incubation at 37° C., 50 μl of solubilization buffer (20% SDS, 66% dimethyl formamide, pH 4.7) were added, and absorbance (570 nm) was measured. Levels of TNF-α release were then calculated from a standard curve obtained using purified human rTNF-α (PeproTech, Inc., Rocky Hill, N.J.). Interferon-γ released by activated T cells was measured by a sandwich enzyme-linked immunosorbent assay. 5×10⁴ γδT cells/well were incubated with stimulus plus 25 units of IL2/well in 100 μl of culture medium during 24 h at 37° C. Then, 50 μl of supernatant were harvested for enzyme-linked immunosorbent assay using mouse monoclonal antibodies (BIOSOURCE, Camarillo, Calif.).

A preferred assay for cytolytic activity is a ⁵¹Cr release assay. In exemplary assays, the cytolytic activity of γδ T cells is measured against autologous normal and tumor target cell lines, or control sensitive target cell lines such as Daudi and control resistant target cell line such as Raji in 4 h ⁵¹Cr release assay. In a specific example, target cells were used in amounts of 2×10³ cells/well and labeled with 100 μCi ⁵¹Cr for 60 minutes. Effector/Target (E/T) ratio ranged from 30:1 to 3.75:1. Specific lysis (expressed as percentage) is calculated using the standard formula [(experimental-spontaneous release/total-spontaneous release)×100].

As discussed, the methods of the invention can generally be carried out with any γδ T cell activator that is capable of stimulating γδ T cell activity. This stimulation can be by direct effect on γδ T cells as discussed below using compounds that can stimulate γδ T cells in a pure γδ T cell culture, or the stimulation can be by an indirect mechanism, such as treatment with pharmacological agents such as statins which prevent biosynthesis of the γδ T cell-stimulating compound isopentenyl pyrophosphate (IPP) or aminobisphosphonates which lead to IPP accumulation (such as, see below). Preferably, however, a γδ T cell activator is a compound capable of regulating the activity of a γδ T cell in a population of γδ T cell clones in culture. The γδ T cell activator is capable of regulating the activity of a γδ T cell population of γδ T cell clones at millimolar concentration, preferably when the γδ T cell activator is present in culture at a concentration of less than 100 mM. Optionally a γδ T cell activator is capable of regulating the activity of a γδ T cell in a population of γδ T cell clones at millimolar concentration, preferably when the γδ T cell activator is present in culture at a concentration of less than 10 mM, or more preferably less than 1 mM. Regulating the activity of a γδ T cell can be assessed by any suitable means, preferably by assessing cytokine secretion, most preferably TNF-α secretion as described herein. Preferably the activator is capable of causing at least a 20%, 50% or greater increase in the number of γδ T cells in culture, or more preferably at least a 2-fold increase in the number of γδ T cells in culture.

In one embodiment, the activator may be a synthetic chemical compound capable of selectively activating Vγ9Vδ2 T lymphocytes. Selective activation of Vγ9Vδ2 T lymphocytes indicates that the compound has a selective action towards specific cell populations, preferably increasing activation of Vγ9Vδ2 T cells at a greater rate or to a greater degree than other T cell types such as Vδ1 T cells, or not substantially not activation other T cell types. Such selectivity can be assessed in vitro T cell activation assays. Such selectivity, as disclosed in the present application, suggests that preferred compounds can cause a selective or targeted activation of the proliferation or biological activity of Vγ9Vδ2 T lymphocytes.

In a preferred embodiment, said γδT cell activator is a compound of the formula I, especially a γδ T cell activator according to formulas I to XVII, especially γδ T cell activator selected from the group consisting of BrHPP, CBrHPP, HDMAPP and epoxPP. However, it will appreciated that a number of less potent γδ T cell activators are available and may be used in accordance with the invention. For example, in one variant, aminobiphosphonate compounds such as pamidronate (Novartis, Nuernberg, Germany) and zoledronate may be used. Other γδ T cell activators for use in the present invention are phosphoantigens disclosed in WO95/20673, isopentenyl pyrophosphate (IPP) (U.S. Pat. No. 5,639,653), as well as alkylamines (such as ethylamine, iso-propyulamine, n-propylamine, n-butylamine and iso-butylamine, for instance). Isobutyl amine and 3-aminopropyl phosphonic acid are obtained from Aldrich (Chicago, Ill.).

Examples of preferred γδ T cell activators according to the present invention comprise the compounds of formula (I): 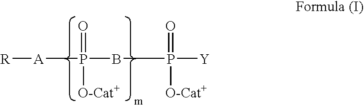
wherein Cat+ represents one (or several, identical or different) organic or mineral cation(s) (including proton);
m is an integer from 1 to 3;
B is O, NH, or any group capable to be hydrolyzed;
Y═O³¹Cat+, a C₁-C₃ alkyl group, a group -A-R, or a radical selected from the group consisting of a nucleoside, an oligonucleotide, a nucleic acid, an amino acid, a peptide, a protein, a monosaccharide, an oligosaccharide, a polysaccharide, a fatty acid, a simple lipid, a complex lipid, a folic acid, a tetrahydrofolic acid, a phosphoric acid, an inositol, a vitamin, a co-enzyme, a flavonoid, an aldehyde, an epoxyde and a halohydrin;
A is O, NH, CHF, CF₂ or CH₂; and,
R is a linear, branched, or cyclic, aromatic or not, saturated or unsaturated, C₁-C₅₀ hydrocarbon group, optionally interrupted by at least one heteroatom, wherein said hydrocarbon group comprises an alkyl, an alkylenyl, or an alkynyl, preferably an alkyl or an alkylene, which can be substituted by one or several substituents selected from the group consisting of: an alkyl, an alkylenyl, an alkynyl, an epoxyalkyl, an aryl, an heterocycle, an alkoxy, an acyl, an alcohol, a carboxylic group (—COOH), an ester, an amine, an amino group (—NH₂), an amide (—CONH₂), an imine, a nitrile, an hydroxyl (—OH), a aldehyde group (—CHO), an halogen, an halogenoalkyl, a thiol (—SH), a thioalkyl, a sulfone, a sulfoxide, and a combination thereof.

In a particular embodiment, the substituents as defined above are substituted by at least one of the substituents as specified above.

Preferably, the substituents are selected from the group consisting of: an (C₁-C₆)alkyl, an (C₂-C₆)alkylenyl, an (C₂-C₆)alkynyl, an (C₂-C₆)epoxyalkyl, an aryl, an heterocycle, an (C₁-C₆)alkoxy, an (C₂-C₆)acyl, an (C₁-C₆)alcohol, a carboxylic group (—COOH), an (C₂-C₆)ester, an (C₁-C₆)amine, an amino group (—NH₂), an amide (—CONH₂), an (C₁-C₆)imine, a nitrile, an hydroxyl (—OH), a aldehyde group (—CHO), an halogen, an (C₁-C₆)halogenoalkyl, a thiol (—SH), a (C₁-C₆)thioalkyl, a (C₁-C₆)sulfone, a (C₁-C₆)sulfoxide, and a combination thereof.

More preferably, the substituents are selected from the group consisting of: an (C₁-C₆)alkyl, an (C₂-C₆)epoxyalkyl, an (C₂-C₆)alkylenyl, an (C₁-C₆)alkoxy, an (C₂-C₆)acyl, an (C₁-C₆)alcohol, an (C₂-C₆)ester, an (C₁-C₆)amine, an (C₁-C₆)imine, an hydroxyl, a aldehyde group, an halogen, an (C₁-C₆)halogenoalkyl, and a combination thereof.

Still more preferably, the substituents are selected from the group consisting of: an (C₃-C₆)epoxyalkyl, an (C₁-C₃)alkoxy, an (C₂-C₃)acyl, an (C₁-C₃)alcohol, an (C₂-C₃)ester, an (C₁-C₃)amine, an (C₁-C₃)imine, an hydroxyl, an halogen, an (C₁-C₃)halogenoalkyl, and a combination thereof, and a combination thereof. Preferably, R is a (C₃-C₂₅)hydrocarbon group, more preferably a (C₅-C₁₀)hydrocarbon group.

In the context of the present invention, the term “alkyl” more specifically means a group such as methyl, ethyl, propyl, isopropyl, butyl, isobutyl, tert-butyl, pentyl, hexyl, heptyl, octyl, nonyl, decyl, undecyl, dodecyl, tridecyl, tetradecyl, pentadecyl, hexadecyl, heptadecyl, octadecyl, nonadecyl, eicosyl, heneicosyl, docosyl and the other isomeric forms thereof. (C₁-C₆)alkyl more specifically means methyl, ethyl, propyl, isopropyl, butyl, isobutyl, tert-butyl, pentyl, hexyl and the other isomeric forms thereof. (C₁-C₃)alkyl more specifically means methyl, ethyl, propyl, or isopropyl.

The term “alkenyl” refers to an alkyl group defined hereinabove having at least one unsaturated ethylene bond and the term “alkynyl” refers to an alkyl group defined hereinabove having at least one unsaturated acetylene bond. (C₂-C₆)alkylene includes a ethenyl, a propenyl (1-propenyl or 2-propenyl), a 1- or 2-methylpropenyl, a butenyl (1-butenyl, 2-butenyl, or 3-butenyl), a methylbutenyl, a 2-ethylpropenyl, a pentenyl (1-pentenyl, 2-pentenyl, 3-pentenyl, 4-pentenyl), an hexenyl (1-hexenyl, 2-hexenyl, 3-hexenyl, 4-hexenyl, 5-hexenyl), and the other isomeric forms thereof. (C₂-C₆)alkynyl includes ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, or 5-hexynyl and the other isomeric forms thereof.

The term “epoxyalkyl” refers to an alkyl group defined hereinabove having an epoxide group. More particularly, (C₂-C₆)epoxyalkyl includes epoxyethyl, epoxypropyl, epoxybutyl, epoxypentyl, epoxyhexyl and the other isomeric forms thereof. (C₂-C₃)epoxyalkyl includes epoxyethyl and epoxypropyl.

The “aryl” groups are mono-, bi- or tri-cyclic aromatic hydrocarbons having from 6 to 18 carbon atoms. Examples include a phenyl, α-naphthyl, β-naphthyl or anthracenyl group, in particular.

“Heterocycle” groups are groups containing 5 to 18 rings comprising one or more heteroatoms, preferably 1 to 5 endocyclic heteroatoms. They may be mono-, bi- or tri-cyclic. They may be aromatic or not. Preferably, and more specifically for R₅, they are aromatic heterocycles. Examples of aromatic heterocycles include pyridine, pyridazine, pyrimidine, pyrazine, furan, thiophene, pyrrole, oxazole, thiazole, isothiazole, imidazole, pyrazole, oxadiazole, triazole, thiadiazole and triazine groups. Examples of bicycles include in particular quinoline, isoquinoline and quinazoline groups (for two 6-membered rings) and indole, benzimidazole, benzoxazole, benzothiazole and indazole (for a 6-membered ring and a 5-membered ring). Nonaromatic heterocycles comprise in particular piperazine, piperidine, etc.

“Alkoxy” groups correspond to the alkyl groups defined hereinabove bonded to the molecule by an —O— (ether) bond. (C₁-C₆)alkoxy includes methoxy, ethoxy, propyloxy, butyloxy, pentyloxy, hexyloxy and the other isomeric forms thereof. (C₁-C₃)alkoxy includes methoxy, ethoxy, propyloxy, and isopropyloxy.

“Alcyl” groups correspond to the alkyl groups defined hereinabove bonded to the molecule by an —CO— (carbonyl) group. (C₂-C₆)acyl includes acetyl, propylacyl, butylacyl, pentylacyl, hexylacyl and the other isomeric forms thereof. (C₂-C₃)acyl includes acetyl, propylacyl and isopropylacyl.

“Alcohol” groups correspond to the alkyl groups defined hereinabove containing at least one hydroxyl group. Alcohol can be primary, secondary or tertiary. (C₁-C₆)alcohol includes methanol, ethanol, propanol, butanol, pentanol, hexanol and the other isomeric forms thereof. (C₁-C₃)alcohol includes methanol, ethanol, propanol and isopropanol.

“Ester” groups correspond to the alkyl groups defined hereinabove bonded to the molecule by an —COO— (ester) bond. (C₂-C₆)ester includes methylester, ethylester, propylester, butylester, pentylester and the other isomeric forms thereof. (C₂-C₃)ester includes methylester and ethylester.

“Amine” groups correspond to the alkyl groups defined hereinabove bonded to the molecule by an —N— (amine) bond. (C₁-C₆)amine includes methylamine, ethylamine, propylamine, butylamine, pentylamine, hexylamine and the other isomeric forms thereof. (C₁-C₃)amine includes methylamine, ethylamine, and propylamine.

“Imine” groups correspond to the alkyl groups defined hereinabove having a (—C═N—) bond. (C₁-C₆)imine includes methylimine, ethylimine, propylimine, butylimine, pentylimine, hexylimine and the other isomeric forms thereof. (C₁-C₃)imine includes methylimine, ethylimine, and propylimine.

The halogen can be Cl, Br, I, or F, more preferably Br or F.

“Halogenoalkyl” groups correspond to the alkyl groups defined hereinabove having at least one halogen. The groups can be monohalogenated or polyhalogenated containing the same or different halogen atoms. For example, the group can be an trifluoroalkyl (CF₃—R). (C₁-C₆)halogenoalkyl includes halogenomethyl, halogenoethyl, halogenopropyl, halogenobutyl, halogenopentyl, halogenohexyl and the other isomeric forms thereof. (C₁-C₃)halogenoalkyl includes halogenomethyl, halogenoethyl, and halogenopropyl.

“Thioalkyl” groups correspond to the alkyl groups defined hereinabove bonded to the molecule by an —S-(thioether) bond. (C₁-C₆)thioalkyl includes thiomethyl, thioethyl, thiopropyl, thiobutyl, thiopentyl, thiohexyl and the other isomeric forms thereof. (C₁-C₃)thioalkyl includes thiomethyl, thioethyl, and thiopropyl.

“Sulfone” groups correspond to the alkyl groups defined hereinabove bonded to the molecule by an —SOO— (sulfone) bond. (C₁-C₆)sulfone includes methylsulfone, ethylsulfone, propylsulfone, butylsulfone, pentylsulfone, hexylsulfone and the other isomeric forms thereof. (C₁-C₃)sulfone includes methylsulfone, ethylsulfone and propylsulfone.

“Sulfoxyde” groups correspond to the alkyl groups defined hereinabove bonded to the molecule by an —SO— (sulfoxide) group. (C₁-C₆)sulfoxide includes methylsulfoxide, ethylsulfoxide, propylsulfoxide, butylsulfoxide, pentylsulfoxide, hexylsulfoxide and the other isomeric forms thereof. (C₁-C₃)sulfoxide includes methylsulfoxide, ethylsulfoxide, propylsulfoxide and isopropylsulfoxide.

“Heteroatom” denotes N, S, or O.

“Nucleoside” includes adenosine, thymine, uridine, cytidine and guanosine.

In a particular embodiment, the hydrocarbon group is a cycloalkylenyl such as a cyclopentadiene or a phenyl, or an heterocycle such as a furan, a pyrrole, a thiophene, a thiazole, an imidazole, a triazole, a pyridine, a pyrimidine, a pyrane, or a pyrazine. Preferably, the cycloalkylenyl or the heterocycle is selected from the group consisting of a cyclopentadiene, a pyrrole or an imidazole. In a preferred embodiment, the cycloalkylenyl or the heterocycle is substituted by an alcohol. Preferably, said alcohol is a (C₁-C₃)alcohol.

In an other embodiment, the hydrocarbon group is an alkylenyl with one or several double bonds. Preferably, the alkylenyl group has one double bond. Preferably, the alkylenyl group is a (C₃-C₁₀)alkylenyl group, more preferably a (C₄-C₇)alkylenyl group. Preferably, said alkylenyl group is substituted by at least one functional group. More preferably, the functional group is selected from the group consisting of an hydroxy, an (C₁-C₃)alkoxy, an aldehyde, an (C₂-C₃)acyl, or an (C₂-C₃)ester. In a more preferred embodiment, the hydrocarbon group is butenyl substituted by a group —CH₂OH. Optionally, said alkenyl group can be the isoform trans (E) or cis (Z), more preferably a trans isoform (E). In a most preferred embodiment, the alkylenyl group is the (E)-4-hydroxy-3-methyl-2-butenyl. In an other preferred embodiment, the alkylenyl group is an isopentenyl, an dimethylallyl or an hydroxydimethylallyl.

In an additional embodiment, the hydrocarbon group is an alkyl group substituted by an acyl. More preferably, the hydrocarbon group is an (C₄-C₇)alkyl group substituted by an (C₁-C₃)acyl.

In a further preferred embodiment, R is selected from the group consisting of: 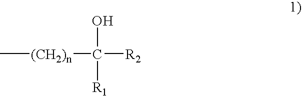
wherein n is an integer from 2 to 20, R₁ is a (C₁-C₃)alkyl group, and R₂ is an halogenated (C₁-C₃)alkyl, a (C₁-C₃)alkoxy-(C₁-C₃)alkyl, an halogenated (C₂-C₃)acyl or a (C₁-C₃)alkoxy-(C₂-C₃)acyl. Preferably, R₁ is a methyl or ethyl group, and R₂ is an halogenated methyl (—CH₂—X, X being an halogen), an halogenated (C₂-C₃)acetyl, or (C₁-C₃)alkoxy-acetyl. The halogenated methyl or acetyl can be mono-, di-, or tri-halogenated. Preferably, n is an integer from 2 to 10, or from 2 to 5. In a more preferred embodiment, n is 2. In a most preferred embodiment, n is 2, R₁ is a methyl and R₂ is an halogenated methyl, more preferably a monohalogenated methyl, still more preferably a bromide methyl. In a particularly preferred embodiment, n is 2, R₁ is a methyl, R2 is a methyl bromide. In a most preferred embodiment, R is 3-(bromomethyl)-3-butanol-1-yl. 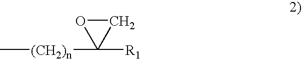
wherein n is an integer from 2 to 20, and R₁ is a methyl or ethyl group. Preferably, n is an integer from 2 to 10, or from 2 to 5. In a more preferred embodiment, n is 2 and R1 is a methyl. 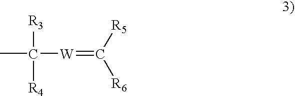
wherein R₃, R₄, and R₅, identical or different, are a hydrogen or (C₁-C₃)alkyl group, W is —CH— or —N—, and R₆ is an (C₂-C₃)acyl, an aldehyde, an (C₁-C₃)alcohol, or an (C₂-C₃)ester. More preferably, R₃ and R₅ are a methyl and R₄ is a hydrogen. More preferably, R₆ is —CH₂—OH, —CHO, —CO—CH₃ or —CO—OCH₃. Optionally, the double-bond between W and C is in conformation trans (E) or cis (Z). More preferably, the double-bond between W and C is in conformation trans (E).

The group Y can allow to design a prodrug. Therefore, Y is enzymolabile group which can be cleaved in particular regions of the subject. The group Y can also be targeting group. In a preferred embodiment, Y is O⁻Cat+, a group -A-R, or a radical selected from the group consisting of a nucleoside, a monosaccharide, an epoxyde and a halohydrin. Preferably, Y is an enzymolabile group. Preferably, Y is O⁻Cat+, a group -A-R, or a nucleoside. In a first preferred embodiment, Y is O⁻Cat+. In a second preferred embodiment, Y is a nucleoside.

In a preferred embodiment, Cat⁺ is H⁺, Na⁺, NH₄⁺, K⁺, Li⁺, (CH₃CH₂)₃NH⁺.

In a preferred embodiment, A is O, CHF, CF₂ or CH₂. More preferably, A is O or CH₂.

In a preferred embodiment, B is O or NH. More preferably, B is O.

In a preferred embodiment, m is 1 or 2. More preferably, m is 1.

In one particular embodiment, synthetic γδT cell activators comprise the compounds of formula (II): 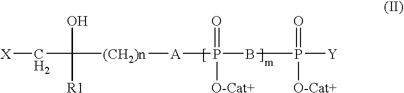
in which X is an halogen (preferably selected from I, Br and Cl), B is O or NH, m is an integer from 1 to 3, R1 is a methyl or ethyl group, Cat+ represents one (or several, identical or different) organic or mineral cation(s) (including the proton), and n is an integer from 2 to 20, A is O, NH, CHF, CF₂ or CH₂, and Y is O⁻Cat+, a nucleoside, or a radical -A-R, wherein R is selected from the group of 1), 2) or 3). Preferably, Y is O⁻Cat+, or a nucleoside. More preferably, Y is O⁻Cat+. Preferably, R1 is a methyl. Preferably, A is O or CH₂. More preferably, A is O. Preferably, n is 2. Preferably, X is a bromide. Preferably, B is O. Preferably, m is 1 or 2. More preferably, m is 1.

For example, synthetic γδT cell activators comprise the compounds of formula (III) or (IV): 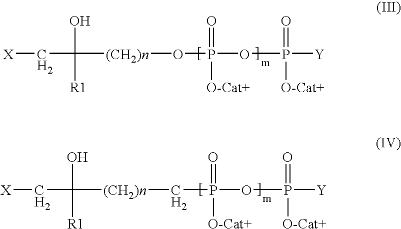
wherein X, R1, n, m and Y have the aforementioned meaning.

In one preferred embodiment, synthetic γδT cell activators comprise the compounds of formula (V): 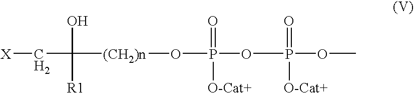
in which X is an halogen (preferably selected from I, Br and Cl), R1 is a methyl or ethyl group, Cat+ represents one (or several, identical or different) organic or mineral cation(s) (including the proton), and n is an integer from 2 to 20. Preferably, R1 is a methyl. Preferably, n is 2. Preferably, X is a bromide.

In a most preferred embodiment, synthetic γδT cell activators comprise the compound of formula (VI):

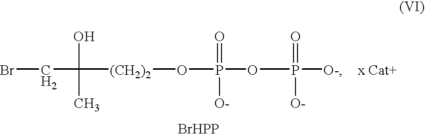

Preferably ×Cat+ is 1 or 2 Na⁺.

In an other most preferred embodiment, synthetic γδT cell activators comprise the compound of formula (VII):

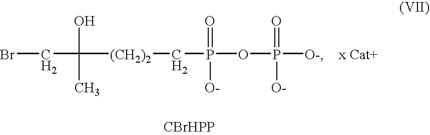

Preferably ×Cat+ is 1 or 2 Na⁺.

In one particular embodiment, synthetic γδT cell activators comprise the compounds of formula (VIII): 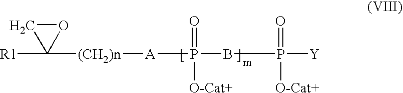
in which R1 is a methyl or ethyl group, Cat+ represents one (or several, identical or different) organic or mineral cation(s) (including the proton), B is O or NH, m is an integer from 1 to 3, and n is an integer from 2 to 20, A is O, NH, CHF, CF₂ or CH₂, and Y is O⁻Cat+, a nucleoside, or a radical -A-R, wherein R is selected from the group of 1), 2) or 3). Preferably, Y is O⁻Cat+, or a nucleoside. More preferably, Y is O⁻Cat+. Preferably, R1 is a methyl. Preferably, A is O or CH₂. More preferably, A is O. Preferably, n is 2. Preferably, B is O. Preferably, m is 1 or 2. More preferably, m is 1.

For example, synthetic γδT cell activators comprise the compounds of formula (IX) or (X): 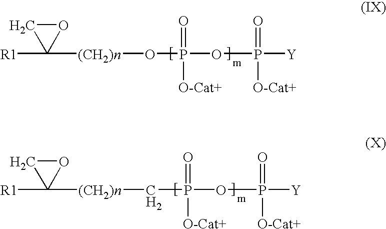
wherein R1, n, m and Y have the above mentioned meaning.

In one preferred embodiment, synthetic γδT cell activators comprise the compounds of formula (XI): 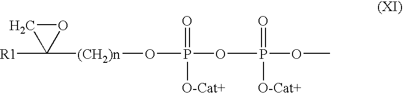
in which R1 is a methyl or ethyl group, Cat+ represents one (or several, identical or different) organic or mineral cation(s) (including the proton), and n is an integer from 2 to 20. Preferably, R1 is a methyl. Preferably, n is 2.

In a most preferred embodiment, synthetic γδT cell activators comprise the compound of formula (XI):

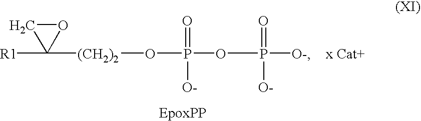

Preferably ×Cat+ is 1 or 2 Na⁺.

In one particular embodiment, synthetic γδT cell activators comprise the compounds of formula (XII): 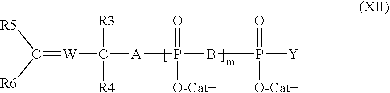
in which R₃, R₄, and R₅, identical or different, are a hydrogen or (C₁-C₃)alkyl group, W is —CH— or —N—, R₆ is an (C₂-C₃)acyl, an aldehyde, an (C₁-C₃)alcohol, or an (C₂-C₃)ester, Cat+ represents one (or several, identical or different) organic or mineral cation(s) (including the proton), B is O or NH, m is an integer from 1 to 3, A is O, NH, CHF, CF₂ or CH₂, and Y is O⁻Cat+, a nucleoside, or a radical -A-R, wherein R is selected from the group of 1), 2) or 3). Preferably, Y is O⁻Cat+, or a nucleoside. More preferably, Y is O⁻Cat+. Preferably, A is O or CH₂. More preferably, A is O. More preferably, R₃ and R₅ are a methyl and R₄ is a hydrogen. More preferably, R₆ is —CH₂—OH, —CHO, —CO—CH₃ or —CO—OCH₃. Preferably, B is O. Preferably, m is 1 or 2. More preferably, m is 1. Optionally, the double-bond between W and C is in conformation trans (E) or cis (Z). More preferably, the double-bond between W and C is in conformation trans (E).

For example, synthetic γδT cell activators comprise the compounds of formula (XIII) or (XIV): 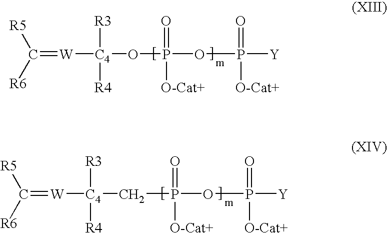
wherein R3, R4, R5, R6, W, m, and Y have the above mentioned meaning. Preferably, W is —CH—. Preferably, R3 and R4 are hydrogen. Preferably, R5 is a methyl. Preferably, R6 is —CH₂—OH.

In a most preferred embodiment, synthetic γδT cell activators comprise the compound of formula (XV):

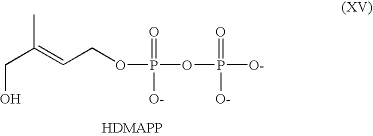

In an other most preferred embodiment, synthetic γδT cell activators comprise the compound of formula (XVI):

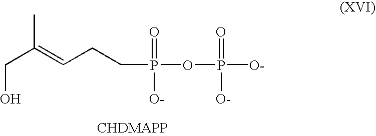

Alternatively, synthetic γδT cell activators can comprise the compound of formula (XVI):

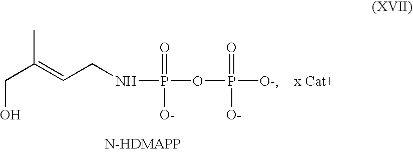

For more details, see international application n^{o} PCT/IB2004/004311, incorporated herein by reference.

Specific examples of compounds include: (E)1-pyrophosphonobuta-1,3-diene; (E)1-pyrophosphonopenta-1,3-diene; (E)1-pyrophosphono-4-methylpenta-1,3-diene; (E,E)1-pyrophosphono-4,8-dimethylnona-1,3,7-triene; (E,E,E)1-pyrophosphono-4,8,12-trimethyltrideca-1,3,7,11-tetraene; (E,E)1-triphosphono-4,8-dimethylnona-1,3,7-triene; 4-triphosphono-2-methylbutene; α,β-di-[3-methylpent-3-enyl]-pyrophosphonate; 1-pyrophosphono-3-methylbut-2-ene; α,γ-di-[3-methylbut-2-enyl]-triphosphonate; α,β-di-[3-methylbut-2-enyl]-pyrophosphonate; allyl-pyrophosphonate; allyl-triphosphonate; α,γ-di-allyl-pyrophosphonate; α,β-di-allyl-triphosphonate; (E,E)4-[(5′-pyrophosphono-6′-methyl-penta-2′,4′-dienyloxymethyl)-phenyl]-phenyl-methanone; (E,E)4-[(5′-triphosphono-6′-methyl-penta-2′,4′-dienyloxymethyl)-phenyl]-phenyl-methanone; (E,E,E)[4-(9′-pyrophosphono-2′,6′-dimethyl-nona-2′,6′,8′-trienyloxymethyl)-phenyl]-phenyl-methanone; (E,E,E)[4-(9′-pyrophosphono-2′,6′,8′-trimethyl-nona-2′,6′,8′-trienyloxymethyl)-phenyl]-phenyl-methanone; 5-pyrophosphono-2-methypentene; 5-triphosphono-2-methypentene; α,γ-di-[4-methylpent-4-enyl]-triphosphonate; 5-pyrophosphono-2-methypent-2-ene; 5-triphosphono-2-methypent-2-ene; 9-pyrophosphono-2,6-dimethynona-2,6-diene; 9-triphosphono-2,6-dimethynona-2,6-diene; α,γ-di-[4,8-dimethylnona-2,6-dienyl]-triphosphonate; 4-pyrophosphono-2-methybutene; 4-methyl-2-oxa-pent-4-enyloxymethylpyrophosphate; 4-methyl-2-oxa-pent-4-enyloxymethyltriphosphate; α,β-di-[4-methyl-2-oxa-pent-4-enyloxymethyl]-pyrophosphate; and α,γ-di-[4-methyl-2-oxa-pent-4-enyloxymethyl]-triphosphate.

In a particular embodiment, the γδT cell activator can be selected from the group consisting of: 3-(halomethyl)-3-butanol-1-yl-diphosphate; 3-(halomethyl)-3-pentanol-1-yl-diphosphate; 4-(halomethyl)-4-pentanol-1-yl-diphosphate; 4-(halomethyl)-4-hexanol-1-yl-diphosphate; 5-(halomethyl)-5-hexanol-1-yl-diphosphate; 5-(halomethyl)-5-heptanol-1-yl-diphosphate; 6-(halomethyl)-6-heptanol-1-yl-diphosphate; 6-(halomethyl)-6-octanol-1-yl-diphosphate; 7-(halomethyl)-7-octanol-1-yl-diphosphate; 7-(halomethyl)-7-nonanol-1-yl-diphosphate; 8-(halomethyl)-8-nonanol-1-yl-diphosphate; 8-(halomethyl)-8-decanol-1-yl-diphosphate; 9-(halomethyl)-9-decanol-1-yl-diphosphate; 9-(halomethyl)-9-undecanol-1-yl-diphosphate; 10-(halomethyl)-10-undecanol-1-yl-diphosphate; 10-(halomethyl)-10-dodecanol-1-yl-diphosphate; 1-(halomethyl)-1-dodecanol-1-yl-diphosphate; 1-(halomethyl)-11-tridecanol-1-yl-diphosphate; 12-(halomethyl)-12-tridecanol-1-yl-diphosphate; 12-(halomethyl)-12-tetradecanol-1-yl-diphosphate; 13-(halomethyl)-13-tetradecanol-1-yl-diphosphate; 13-(halomethyl)-13-pentadecanol-1-yl-diphosphate; 14-(halomethyl)-14-pentadecanol-1-yl-diphosphate; 14-(halomethyl)-14-hexadecanol-1-yl-diphosphate; 15-(halomethyl)-15-hexadecanol-1-yl-diphosphate; 15-(halomethyl)-15-heptadecanol-1-yl-diphosphate; 16-(halomethyl)-16-heptadecanol-1-yl-diphosphate; 16-(halomethyl)-16-octadecanol-1-yl-diphosphate; 17-(halomethyl)-17-octadecanol-1-yl-diphosphate; 17-(halomethyl)-17-nonadecanol-1-yl-diphosphate; 18-(halomethyl)-18-nonadecanol-1-yl-diphosphate; 18-(halomethyl)-18-eicosanol-1-yl-diphosphate; 19-(halomethyl)-19-eicosanol-1-yl-diphosphate; 19-(halomethyl)-19-heneicosanol-1-yl-diphosphate; 20-(halomethyl)-20-heneicosanol-1-yl-diphosphate; 20-(halomethyl)-20-docosanol-1-yl-diphosphate; 21-(halomethyl)-21-docosanol-1-yl-diphosphate; and 21-(halomethyl)-21-tricosanol-1-yl-diphosphate.

More particularly, the γδT cell activator can be selected from the group consisting of: 3-(bromomethyl)-3-butanol-1-yl-diphosphate (BrHPP); 5-bromo-4-hydroxy-4-methylpentyl pyrophosphonate (CBrHPP); 3-(iodomethyl)-3-butanol-1-yl-diphosphate (IHPP); 3-(chloromethyl)-3-butanol-1-yl-diphosphate (ClHPP); 3-(bromomethyl)-3-butanol-1-yl-triphosphate (BrHPPP); 3-(iodomethyl)-3-butanol-1-yl-triphosphate (IHPPP); α,γ-di-[3-(bromomethyl)-3-butanol-1-yl]-triphosphate (diBrHTP); and α,γ-di-[3-(iodomethyl)-3-butanol-1-yl]-triphosphate (diIHTP).

In an other particular embodiment, the γδT cell activator can be selected from the group consisting of: 3,4-epoxy-3-methyl-1-butyl-diphosphate (Epox-PP); 3,4,-epoxy-3-methyl-1-butyl-triphosphate (Epox-PPP); α,γ-di-3,4,-epoxy-3-methyl-1-butyl-triphosphate (di-Epox-TP); 3,4-epoxy-3-ethyl-1-butyl-diphosphate; 4,5-epoxy-4-methyl-1-pentyl-diphosphate; 4,5-epoxy-4-ethyl-1-pentyl-diphosphate; 5,6-epoxy-5-methyl-1-hexyl-diphosphate; 5,6-epoxy-5-ethyl-1-hexyl-diphosphate; 6,7-epoxy-6-methyl-1-heptyl-diphosphate; 6,7-epoxy-6-ethyl-1-heptyl-diphosphate; 7,8-epoxy-7-methyl-1-octyl-diphosphate; 7,8-epoxy-7-ethyl-1-octyl-diphosphate; 8,9-epoxy-8-methyl-1-nonyl-diphosphate; 8,9-epoxy-8-ethyl-1-nonyl-diphosphate; 9,10-epoxy-9-methyl-1-decyl-diphosphate; 9,10-epoxy-9-ethyl-1-decyl-diphosphate; 10,11-epoxy-10-methyl-1-undecyl-diphosphate; 10,11-epoxy-10-ethyl-1-undecyl-diphosphate; 11,12-epoxy-11-methyl-1-dodecyl-diphosphate; 11,12-epoxy-11-ethyl-1-dodecyl-diphosphate; 12,13-epoxy-12-methyl-1-tridecyl-diphosphate; 12,13-epoxy-12-ethyl-1-tridecyl-diphosphate; 13,14-epoxy-13-methyl-1-tetradecyl-diphosphate; 13,14-epoxy-13-ethyl-1-tetradecyl-diphosphate; 14,15-epoxy-14-methyl-1-pentadecyl-diphosphate; 14,15-epoxy-14-ethyl-1-pentadecyl-diphosphate; 15,16-epoxy-15-methyl-1-hexadecyl-diphosphate; 15,16-epoxy-15-ethyl-1-hexadecyl-diphosphate; 16,17-epoxy-16-methyl-1-heptadecyl-diphosphate; 16,17-epoxy-16-ethyl-1-heptadecyl-diphosphate; 17,18-epoxy-17-methyl-1-octadecyl-diphosphate; 17,18-epoxy-17-ethyl-1-octadecyl-diphosphate; 18,19-epoxy-18-methyl-1-nonadecyl-diphosphate; 18,19-epoxy-18-ethyl-1-nonadecyl-diphosphate; 19,20-epoxy-19-methyl-1-eicosyl-diphosphate; 19,20-epoxy-19-ethyl-1-eicosyl-diphosphate; 20,21-epoxy-20-methyl-1-heneicosyl-diphosphate; 20,21-epoxy-20-ethyl-1-heneicosyl-diphosphate 21,22-epoxy-21-methyl-1-docosyl-diphosphate; and 21,22-epoxy-21-ethyl-1-docosyl-diphosphate.

In a further particular embodiment, the γδT cell activator can be selected from the group consisting of: 3,4-epoxy-3-methyl-1-butyl-diphosphate (Epox-PP); 3,4,-epoxy-3-methyl-1-butyl-triphosphate (Epox-PPP); α,γ-di-3,4,-epoxy-3-methyl-1-butyl-triphosphate (di-Epox-TP); and uridine 5′-triphosphate-(3,4-époxy methyl butyl) (Epox-UTP).

In a preferred embodiment, the γδT cell activator can be selected from the group consisting of: (E)-4-hydroxy-3-methyl-2-butenyl pyrophosphate (HDMAPP) and (E)-5-hydroxy-4-methylpent-3-enyl pyrophosphonate (CHDMAPP).

These compounds may be produced according to various techniques known per se in the art, some of which being disclosed in PCT Publications nos. WO 00/12516, WO 00/12519, WO 03/050128, and WO 03/009855, the disclosures of which are incorporated herein by reference.

In a most preferred embodiment, the γδT cell activator is selected from the group consisting of HDMAPP, CHDMAPP, Epox-PP, BrHPP and CBrHPP, more preferably HDMAPP, CHDMAPP, BrHPP and CBrHPP, still more preferably HDMAPP.

Alternatively, although potentially less efficient, other activators for use in the present invention are phosphoantigens disclosed in WO 95/20673, isopentenyl pyrophosphate (IPP) (U.S. Pat. No. 5,639,653) and 3-methylbut-3-enyl pyrophosphonate (C—IPP). The disclosures of both references are incorporated herein by reference.

Compounds comprising a nucleoside as Y group can be prepared, for example, by the following reactions. Depending on the type and reactivity of the functional groups provided by Y, the professional is able to adapt the following examples, if necessary including the phases of protection/non-protection of the sensitive functional groups or those that can interact with the coupling reaction. 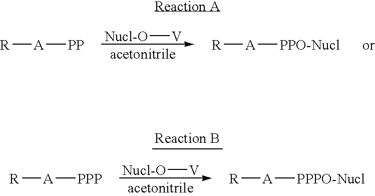
where —O—V is a good group beginning with V chosen, for example, from among tosyle, mesyle, triflyle, brosyle or bromium, PP represents the pyrophosphate group, PPP represents the triphosphate group, R-A- has the above mentioned meaning and Nucl is a nucleoside. Preferably, Nucl-O—V is selected from the group consisting of: 5′-O-Tosyladenosine, 5′-O-Tosyluridine, 5′-O-Tosylcytidine, 5′-O-Tosylthymidine or 5′-O-Tosyl-2′-deoxyadenosine.

For example, for the compound with R of group 1), the reaction procedure can be the following: 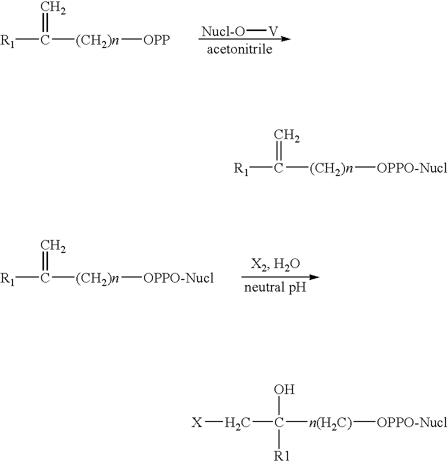
where —O—V is a good group beginning with V chosen, for example, from among tosyle, mesyle, triflyle, brosyle or bromium, PP represents the pyrophosphate group and Nucl is a nucleoside. Preferably, Nucl-O—V is selected from the group consisting of: 5′-O-Tosyladenosine, 5′-O-Tosyluridine, 5′-O-Tosylcytidine, 5′-O-Tosylthymidine or 5′-O-Tosyl-2′-deoxyadenosine as described in Davisson et al, (1987), the disclosure of which is incorporated herein by reference.

Neutral pH is a nucleophile substitution reaction that can be carried out in conditions similar to those described by Davisson et al, (1987); and Davisson et al. (1986), the disclosures of which are incorporated herein by reference.

This reaction can also be used to prepare compound comprising a monosaccharide as group Y. In this case, Nucl-O—V is replaced by MonoSac-O—V, wherein Monosac is monosaccharide. For example, it is possible to use the MonoSac-O—Y group corresponding to compound Methyl-6-O-tosyl-alpha-D-galactopyranoside as described in publication Nilsson and Mosbach, (1980), incorporated herein by reference, or the commercially available mannose triflate compound.

This reaction can further be used to prepare compound comprising a oligosaccharide as group Y. In this case, Nucl-O—V is replaced by oligoSac-O—V, wherein oligoSac is an oligosaccharide. For example, it is possible to use the oligoSac-O—Y group corresponding to compound 6^{A}-O-p-Toluenesulfonyl-β-cyclodextrin as described in publication (Organic syntheses, Vol. 77, p 225-228, the disclosure of which is incorporated herein by reference).

This reaction can be used to prepare compound comprising a polysaccharide as group Y. In this case, Nucl-O—V is replaced by polySac-O—V, wherein polySac is a polysaccharide. For example, it is possible to use the polySac-O—Y group corresponding to tosylated polysaccharide as described in publication Nilsson et al., (1981); and Nilsson and Mosbach, (1980), the disclosures of which are incorporated herein by reference. This coupling technique based on the activation of the hydroxyl groups of a polysaccharide support by tosylation allows for covalent coupling in an aqueous or an organic medium.

This reaction can also be used for preparing compound comprising an aldehyde derivative as group Y by choosing, instead of Nucl, a derivative including a protected aldehyde function in the form of an acetal or any other group protecting this function.

Alternatively, compounds comprising a nucleoside as Y group can be prepared by the following reaction: 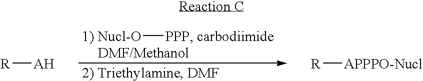
where PPP represents the triphosphate group, R-A has the above mentioned meaning, DMF is dimethylformamide, and Nucl is a nucleoside. This reaction can be carried out in conditions similar to those described by Knorre et al. (1976), or by Bloom et al., U.S. Pat. No. 5,639,653 (1997), the disclosures of which are incorporated herein by reference, from alcohol and a nucleotide with formula Nucl-O—PPP.

For example, for the compound with R of group 1), the reaction procedure can be the following: 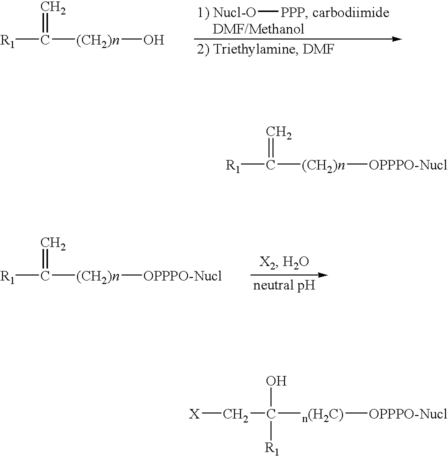
where PPP represents the triphosphate group, DMF is dimethylformamide, and Nucl is a nucleoside.

This reaction can also be applied to the preparation of oligonucleotides 5′-triphosphate ?-esters as indicated by the authors of publication Knorre et al. (1976).

Compounds comprising a nucleic acid as Y group, more particularly a ribonucleic acid, can be prepared in conditions similar to those described in publication F. Huang et al (1997). The authors describe a universal method from catalytic RNA that is applicable to any molecule comprising a free terminal phosphate group. Compounds structurally related to the phosphohalohydrine group such as isopentenyl pyrophosphate or thiamine pyrophosphate are used or mentioned by these authors (see p. 8968 of F. Huang et al (1997)). It should also be noted that the experimental conditions for the coupling procedure (in particular pH conditions) described in the section <<Reaction of Isolate 6 pppRNA with phosphate containing Nucleophiles>> on page 8965 are compatible with the presence of a halohydrine function.

Compounds comprising an amino acid, a peptide or a protein derivative as Y group can be obtained using the well known reactivity of their primary amine or thiol function on an epoxyde function (S_{N}2 reaction). This type of coupling classically involves an intermediate group still called “linker” bearing an epoxyde function. An example of a reaction procedure using this type of coupling is provided below. 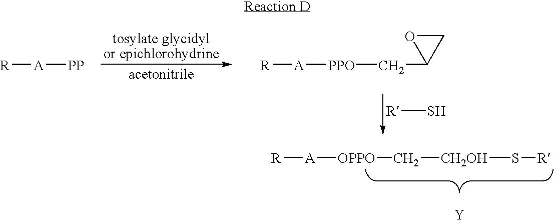
where PP represents the pyrophosphate group, R-A has the above mentioned meaning and R′—SH is an amino acid, a peptide or a protein derivative. The first phase can be carried out in conditions similar to those described by Davisson et al. (1987) and Davisson et al, (1986), the disclosures of which are incorporated herein by reference, from the tetrabutylammonium salt of the initial compound and commercially available compounds such as glycidyl tosylate or epichlorohydrine. This reaction can also be carried out with thriphosphate compounds. Alternatively, a primary amine R′—NH₂ can be used instead of R′—SH. Without the reaction with R′—SH, the first reaction can be used to prepare compound comprising an epoxyde derivative.

Alternatively, compounds comprising an amino acid, a peptide or a protein derivative as Y group can be prepared by the following reaction: 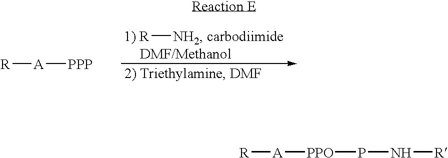
where PPP represents the triphosphate group, PP represents the pyrophosphate group, P represents the phosphate group, R-A has the above mentioned meaning and R′—NH is an amino acid, a peptide or a protein derivative. The reaction can be carried out in conditions similar to those described by Knorre et al. (1976), the disclosure of which is incorporated herein by reference, from compound (R-A-PPP) and an amino acid, peptide or a protein with formula R—NH₂. This reaction involves the protection of the sensitive functions of compound R—NH₂ or can react with the carbodiimide (in particular, the carboxyl function).

Tri or tetra-n-butylammonium salts of phosphoric, pyrophosphoric, triphosphoric, tetra-phosphoric or polyphosphoric acid can be prepared from commercially available corresponding acids. Derivatives with a related structure such as derivatives of methanetrisphosphonic acid described in publication Liu et al (1999), the disclosure of which is incorporated herein by reference, can also be prepared according to the reaction procedure. The above mentioned reactions can be extrapolated to a very large spectrum of molecules or biomolecules by using the reactivity of the hydroxyl, amine, phosphate or thiol functions. Thereby, inositol derivatives can be prepared according to reactions A or B by activation of the hydroxyl function. Derivatives of folic acid (vitamin B9) or tetrahydrofolic acid can be prepared according to reactions D or E by calling on the reactivity of the primary amine function.

Of course, other types of coupling can be considered and the professional can have access to a large choice of reactions.

Thereby, coupling by phosphorylation of carboxylic acid or phenol groups can be used for the formation of fatty acid, lipid or certain flavonoid derivatives.

Each of the foregoing references relating to compounds and their synthesis are incorporated herein by reference.

As discussed, preferred compounds are selected which increase the biological activity of γδ T cells, preferably increasing the activation of γδ T cells, particularly increasing cytokine secretion from γδ T cells or increasing the cytolytic activity of γδ T cells, with or without also stimulating the expansion of γδ T cells. For example, a γδ T cell activator allows the cytokine secretion by γδ T cells to be increased at least 2, 3, 4, 10, 50, 100-fold, as determined in vitro. Cytokine secretion and cytolytic activity can be assessed using any appropriate in vitro assay, or those described herein.

In another aspect, the present invention relates to methods for the treatment of a carcinoma or viral infection, preferably a urinary or bladder cancer or an HPV infection, where the γδ T cell activator is administered in an amount and under conditions sufficient to stimulate the expansion of the γδ T cell population in a subject, particularly to reach 30-90% of total circulating lymphocytes, typically 40-90%, more preferably from 50-90%. In typical embodiments, the invention allows the selective expansion of γδ T cells in a subject, to reach at least 20%, 30% or 40% of total circulating lymphocytes. Percentage of total circulating lymphocytes can be determined according to methods known in the art. A preferred method for determining the percentage of γδ T cells in total circulating lymphocytes is by flow cytometry.

In another aspect, the present invention relates to methods for the treatment of a carcinoma or viral infection, preferably a urinary or bladder cancer or an HPV infection, where the γδ T cell activator is administered in an amount and under conditions sufficient to stimulate the expansion of the γδ T cell population in a subject, particularly to increase by more than 2-fold the number of γδ T cells in a subject, typically at least 10-fold, more preferably at least 20-fold. In another aspect, the present invention relates to methods for the treatment of a bladder cancer, where the γδ T cell activator, especially a γδ T cell activator according to formulas I to XVII, is administered in an amount and under conditions sufficient to stimulate the expansion of the γδ T cell population in a subject, particularly to reach a circulating γδ T cell count of at least 500 γδ T cells/mm3 in a subject, typically at least 1000 γδ T cells/mm3, more preferably at least 2000 γδ T cells/mm3. The number of γδ T cells and circulating γδ T cell count in a subject is preferably assessed by obtaining a blood sample from a patient before and after administration of said γδ T cell activator and determining the difference in number of γδ T cells present in the sample.

Preferably, dosage (single administration) of a γδ T cell activator compound of formula I for treatment is between about 1 μg/kg and about 1.2 g/kg. It will be appreciated that the above dosages related to a group of compounds, and that each particular compound may vary in optimal doses, as further described herein for exemplary compounds. Nevertheless, compounds are preferably administered in a dose sufficient to significantly increase the biological activity of γδ T cells or to significantly increase the γδ T cell population in a subject. Said dose is preferably administered to the human by intravenous (i.v.) administration during 2 to 180 min, preferably 2 to 120 min, more preferably during about 5 to about 60 min, or most preferably during about 30 min or during about 60 min. In preferred exemplary compounds, a compound of formula II to XI is administered in a dosage (single administration) between about 0.1 mg/kg and about 1.2 g/kg, preferably between about 10 mg/kg and about 1.2 g/kg, more preferably between about 5 mg/kg and about 100 mg/kg, even more preferably between about 5 μg/kg and 60 mg/kg. Most preferably, dosage (single administration) for three-weekly or four-weekly treatment (treatment every three weeks or every third week) is between about 0.1 mg/kg and about 1.2 g/kg, preferably between about 10 mg/kg and about 1.2 g/kg, more preferably between about 5 mg/kg and about 100 mg/kg, even more preferably between about 5 μg/kg and 60 mg/kg. In preferred exemplary compounds, a compound of formula XII to XVII, is administered in a dosage (single administration) between about 1 μg/kg and about 100 mg/kg, preferably between about 10 μg/kg and about 20 mg/kg, more preferably between about 20 μg/kg and about 5 mg/kg, even more preferably between about 20 μg/kg and 2.5 mg/kg. Most preferably, dosage (single administration) for three-weekly or four-weekly treatment (treatment every three weeks or every third week) is between about 1 μg/kg and about 100 mg/kg, preferably between about 10 μg/kg and about 20 mg/kg, more preferably between about 20 μg/kg and about 5 mg/kg, even more preferably between about 20 μg/kg and 2.5 mg/kg. Further detail on dosages and administration and examples of dose response experiments using γδ T cell activator in mice and primate models are provided in co-pending PCT Application no. PCT/FR03/03560 filed 2 Dec. 2003, the disclosure of which is incorporated herein by reference.

Pharmaceutical Formulations of γδT Cell Activator Compounds, IMC and IC Compounds

It will be appreciated that active compounds for use in the invention—e.g. IMC and IC compounds of the invention, as well as the γδT cell activator compounds—can be administered by any suitable routes of administration including, but not limited to, oral, dermal, subcutaneous, percutaneous, intramuscular, intraperitoneal, intravenous, intradermal, intrathecal, intralesional, intratumoral, intrabladder, intra-vaginal, intraocular, intrarectal, intrapulmonary, intraspinal, transdermal, subdermal, placement within cavities of the body, nasal inhalation, pulmonary inhalation, impression into skin and electrocorporation.

In the context of the present invention the term “intratumoral administration” means that the composition is delivered directly into the tumor, i.e. into actively dividing tumor cells surrounding the necrotic central part of the tumor and not, e.g., only into peritumoral cells or into the center of the tumor. The term “tumor” in this context does not only refer to the primary tumor but also to metastases. Appropriate means for intratumoral administration are, e.g., injection, ballistic tools, electroporation, electroinsertion, wounding, scratching, pressurized insertion tools, dermojets, etc. In a preferred embodiment, intra-tumoral administration is carried out by injection, preferably by a needle and a syringe.

In the context of administering a composition to an individual, the terms “local administration” and “topical administration” are used interchangeably, and refer to administration of a composition to a definite place or locality on the individual's body.

It will be appreciated that active compounds for use in the invention—e.g. IMC and IC compounds of the invention, as well as the γδT cell activator compounds—can be incorporated into pharmaceutical compositions suitable for administration. Such compositions typically comprise a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradernial, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose, pH can be adjusted with acids or bases, such as hydrochloric acid or sodium hydroxide. The parenteral preparation can be enclosed in ampoules, disposable syringes or multiple dose vials made of glass or plastic. Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi.

The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungat agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition.

Prolonged absorption of the injectable compositions can be brought about by including in the composition an agent which delays absorption, for example, aluminum nionostearate and gelatin.

Where the compound is a protein, peptide or antibody, sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. For administration by inhalation, the compounds are delivered in the form of an aerosol spray from pressured container or dispenser which contains a suitable propellant, e.g., a gas such as carbon dioxide, or a nebulizer. Systemic administration can also be by transmucosal or transdernial means. For transmucosal or transdernial administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art. Most preferably, active compound is delivered to a subject by intravenous injection.

Administration to skin can involve dermal or transdermal administration according to any suitable method, a number of methods being available in the art. A composition to be administered will often comprise the physiologically active agent together with a penetration enhancer incorporated into a dosage form for topical application to the skin or mucous membranes of animals. Suitable dosage forms include creams, lotions, gels, ointments, suppositories, mousses, spray, for example nasal sprays, aerosols, buccal and sublingual tablets, gingival and buccal patches or any one of a variety of transdermal devices for use in the continuous administration of systematically active drugs by absorption through the skin, oral mucosa or other membranes. Some examples of suitable vehicles are given in U.S. Pat. Nos. 3,598,122, 3,598,123, 3,742,951, 3,814,097, 3,921,636, 3,993,072, 3,993,073, 3,996,934, 4,031,894, 4,060,084, 4,069,307, 4,201,211, 4,230,105, 4,292,299, 4,292,303, 5,323,769, 5,023,085, 5,474,783, 4,941,880 and U.S. Pat. No. 4,077,407. These patents also disclose a variety of specific systematically active agents which may also be useful in transdermal delivery in adjunct to those of this invention. These disclosures are thus hereby incorporated herein by reference

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or preferably parenteral compositions in dosage unit form for case of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Exemplary Treatment Regimens

Bladder Cancer

Treatment with a mycobacterial antigen and a γδ T cell activator can be carried out according to any suitable administration regimen. The γδ T cell activator may be administered through any of several different routes, typically by injection or oral administration. Injection may be carried out into various tissues, such as by intravenous, intra-peritoneal, intra-arterial, intra-muscular, intra-dermic, subcutaneous, etc. Particularly preferred is intravenous injection. The γδ T cell activator can be administered before, at the same time or after the mycobacterial antigen is administered. Generally, the γδ T cell activator will be administered no more than several (4, 5, 6, or 7) days before or after treatment with the mycobacterial antigen. Most preferably, however, the γδ T cell activator is administered at substantially the same time as the mycobacterial antigen is administered, preferably within 48 hours, 24 hours or more preferably within 12 or within 6 hours of treatment with the mycobacterial antigen. For example, in the regimen of the Examples, the γδ T cell activator is administered several hours before administration of the mycobacterial antigen.

In one aspect, the γδ T cell activator is administered once during the course of mycobacterial antigen therapy. More preferably, however, the γδ T cell activator is administered several times. Most preferably, the γδ T cell activator is administered according to a regimen in which γδ T cell activity, preferably the γδ T cell rate (number of γδ T cells), is allowed to return to substantially basal rate prior to a second administration of the compound. As provided in co-pending PCT Application no. PCT/FR03/03560 filed 2 Dec. 2003, the disclosure of which is incorporated herein by reference, at least about one week, but more preferably at least about two weeks, are required for a patient's γδ T cell rate to return to substantially basal rate.

The course of a preferred cycle for administering the γδ T cell activator is an at least 1-weekly cycle, but more preferably at least a 2-weekly cycle (at least about 14 days), or more preferably at least 3-weekly or 4-weekly, though cycles anywhere between 2-weekly and 4-weekly are preferred. Also effective and contemplated are cycles of up to 8-weekly, for example 5-weekly, 6-weekly, 7-weekly or 8-weekly. In a preferred embodiment, the γδ T cell activator is administered only the first day of a 2-weekly to 4-weekly, or preferably 3 weekly, cycle.

In an exemplary embodiment, the mycobacterial antigen is administered on a 1-weekly cycle for 6 weeks, and the γδ T cell activator occurs on the first day of a 2-weekly to 4-weekly cycle (that is, an about 14 to 28 day weeks repeating cycle). In a preferred embodiment, the mycobacterial antigen is administered on a 1-weekly cycle for 6 weeks and the γδ T cell activator is administered only the first day of the 2-weekly to 4-weekly, or preferably 3 weekly, cycle.

Preferably the γδ T cell activator is administered for at least substantially the duration of mycobacterial antigen treatment. For example, a 3-weekly cycle is used for the γδ T cell activator and a 1-weekly cycle is used for the mycobacterial antigen, both over a course of six weeks according to the following scheme:
Day 0: mycobacterial antigen and γδ T cell activator
Day 7: mycobacterial antigen
Day 14: mycobacterial antigen
Day 21: mycobacterial antigen and γδ T cell activator
Day 28: mycobacterial antigen
Day 35: mycobacterial antigen
Day 42: (optional): γδ T cell activator

In other exemplary administration regimens, particularly in maintenance therapy, a 3-weekly cycle is used for both the γδ T cell activator and the mycobacterial antigen. Preferably the γδ T cell activator and the mycobacterial antigen are administered on the same day.

As mentioned, a subject will preferably be treated for at least two cycles of γδ T cell activator, or more preferably for at least three cycles, or for at least one cycle of mycobacterial antigen therapy, preferably at least one cycle of 1-weekly mycobacterial antigen administration for a 6 week treatment cycle. In other aspect, treatment may continue for a greater number of cycles, for example at least 4, 5, 6 or more cycles can be envisioned. At the end of each cycle, the cycle of dosing may be repeated for as long as clinically tolerated and the tumor is under control or until tumor regression. In exemplary mycobacterial antigen regimens, administrations of mycobacterial antigen take place 1-weekly for 6 weeks, followed by an interval (for example 6 weeks), followed by administrations of mycobacterial antigen 3-weekly for a desired duration, such as at least 6 months or 12 months for maintenance therapy.

In other embodiments, the methods of the invention comprises further administering a cytokine. While the compounds of the invention may be used with or without further administration, in a preferred aspect a cytokine can be administered, wherein said cytokine is capable of increasing the expansion of a γδ T cell population treated with a γδ T cell activator compound, preferably wherein the cytokine is capable of inducing an expansion of a γδ T cell population which is greater than the expansion resulting from administration of the γδ T cell activator compound in the absence of said cytokine. A preferred cytokine is an interleukin-2 polypeptide.

A cytokine having γδ T cell proliferation inducing activity, most preferably the interleukin-2 polypeptide, is administered at low doses, typically over a period of time comprised between 1 and 10 days. The γδ T cell activator is preferably administered in a single dose, and typically at the beginning of a cycle.

In preferred aspects, a cytokine, most preferably IL-2, is administered daily for up to about 10 days, preferably for a period of between about 3 and 10 days, or most preferably for about 7 days. Preferably, the administration of the cytokine begins on the same day (e.g. within 24 hours of) as administration of the γδ T cell activator. It will be appreciated that the cytokine can be administered in any suitable scheme within said regimen of between about 3 and 10 days. For example, in one aspect the cytokine is administered each day, while in other aspects the cytokine need not be administered on each day.

HPV Infection

Infections caused by human papilloma virus (HPV) using the Mycobacterium that can be treated according to the methods of the invention may include cutaneous and genital warts in humans, including verruca vulgaris and condyloma acurninaturn, cervical intraepithelial neoplasia and genital carcinomas. In general, the treatment is applicable to any disease condition caused by HPV in humans including penile, intraurethral, perianal, intra-anal or perineal infections in men and cervical, vaginal, perigenital, intra-urethral, intra-anal and perineal infections in women, including condylomata acuminata, penile cancer, Bowen's disease, cervical cancer, head and neck cancer, laryngeal papillomatosis and laryngeal carcinoma.

The mycobacterial antigen treatment may be effected by application of the Mycobacterium antigen in a suitable carrier to the region of infection, which may involve topical application to cutaneous, penile and perianal areas, or intraurethral application to the urogenital tract. The treatment may involve a single or a plurality of doses applied at time intervals. The individual dosage level may be about 1 mg to about 500 mg attenuated BCG while the time interval between doses may vary from about 1 to about 30 days. The number of treatments applied is from 1 to about 30 treatments. The mycobacterial antigen and γδ T cell activator combination treatment is can be used alone or may be preceded by laser or other surgical or topical therapy.

The γδ T cell activator may be administered as for the treatment of bladder carcinoma, such as through any of several different routes, typically by injection or oral administration. Injection may be carried out into various tissues, such as by intravenous, intra-peritoneal, intra-arterial, intra-muscular, intra-dermic, subcutaneous, etc. Particularly preferred is intravenous injection. The γδ T cell activator can be administered before, at the same time or after the mycobacterial antigen is administered. Generally, the γδ T cell activator will be administered no more than several (4, 5, 6, or 7) days before or after treatment with the mycobacterial antigen. Most preferably, however, the γδ T cell activator is administered at substantially the same time as the mycobacterial antigen is administered, preferably within 48 hours, 24 hours or more preferably 12 or 6 hours of treatment with the mycobacterial antigen. For example, in the regimen of the Examples, the γδ T cell activator is administered several hours before administration of the mycobacterial antigen.

In one aspect, the γδ T cell activator is administered once during the course of mycobacterial antigen therapy. More preferably, however, the γδ T cell activator is administered several times. Most preferably, the γδ T cell activator is administered according to a regimen in which γδ T cell activity, preferably the γδ T cell rate (number of γδ T cells), is allowed to return to substantially basal rate prior to a second administration of the compound. The course of a preferred cycle for administering the γδ T cell activator is an at least 1-weekly cycle, but more preferably at least a 2-weekly cycle (at least about 14 days), or more preferably at least 3-weekly or 4-weekly, though cycles anywhere between 2-weekly and 4-weekly are preferred. Also effective and contemplated are cycles of up to 8-weekly, for example 5-weekly, 6-weekly, 7-weekly or 8-weekly. In a preferred embodiment, the γδ T cell activator is administered only the first day of a 2-weekly to 4-weekly, or preferably 3 weekly, cycle.

In an exemplary embodiment, the mycobacterial antigen is administered on a 1-weekly cycle for about 6 weeks, or for at least 4, 6, 8, 10 or 12 weeks, and the γδ T cell activator occurs on the first day of a 2-weekly to 4-weekly cycle (that is, an about 14 to 28 day weeks repeating cycle). In a preferred embodiment, the mycobacterial antigen is administered on a 1-weekly cycle for 6 weeks and the γδ T cell activator is administered only the first day of the 2-weekly to 4-weekly, or preferably 3 weekly, cycle. Preferably the γδ T cell activator is administered for at least substantially the duration of mycobacterial antigen treatment. For example, a 3-weekly cycle is used for the γδ T cell activator and a 1-weekly cycle is used for the mycobacterial antigen, both over a course of six weeks according to the following scheme:
Day 0: mycobacterial antigen and γδ T cell activator
Day 7: mycobacterial antigen
Day 14: mycobacterial antigen
Day 21: mycobacterial antigen and γδ T cell activator
Day 28: mycobacterial antigen
Day 35: mycobacterial antigen
Day 42: (optional): γδ T cell activator

In other exemplary administration regimens, a 3-weekly cycle is used for both the γδ T cell activator and the mycobacterial antigen. Preferably the γδ T cell activator and the mycobacterial antigen are administered on the same day. In other embodiments, a cytokine may additionally be administered, according to a regimen as described for the treatment of bladder carcinoma.

For topical use in the treatment of HPV infection, the Mycobacterium may be formulated with a keratolytic agent for topical application to the region of infection, particularly as a cream for adherent application to the region of infection. The keratolytic agent may be salicylic acid, which may be powdered. The keratolytic agent may be present in an amount of about 0.1 to about 50 wt %, preferably about 1 to about 10 wt %. The composition which is applied to the area of infection may take any desired form, for example, a cream, a powder or ointment. Any desired form of application may be employed, including slow-release systems, plasters and transdermal systems.

The following examples will serve to further illustrate the present invention without, at the same time, however, constituting any limitation thereof. On the contrary, it is to be clearly understood that resort may be had to various other embodiments, modifications, and equivalents thereof which, after reading the description herein, may suggest themselves to those skilled in the artwithout departing from the spirit of the present invention and/or the scope of the appended claims.

### EXAMPLES

### Example 1

### Synthesis of HDMAPP

(E)-4-Hydroxy-3-methylbut-2-enyl diphosphate is prepared according to the method of Wolff et al, Tetrahedron Letters (2002) 43:2555 or Hecht et al, Tetrahedron Letters (2002) 43: 8929. For the purpose of performing biological testing, the aqueous solutions of the product are sterilized by filtration through a 0.2 μm filter and stored at −20° C. In the case of testing performed in vivo, the solutions are passed beforehand through a DOWEX 50WX8-200 cationic resin column (sodium form) eluted by two column volumes of deionized water.

### Example 2

### Synthesis of C-HDMAPP

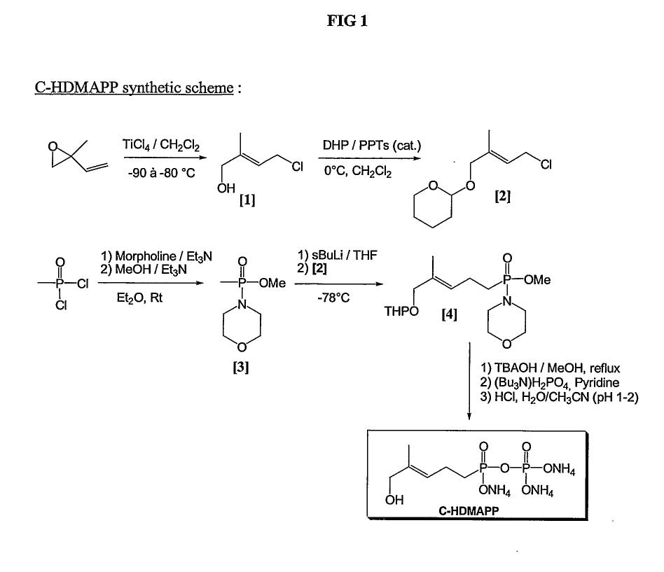

C-HDMAPP synthesis is carried out as follows, the scheme for which is also shown in FIG. 1. References in Example 2 are made to FIG. 1 by showing the reference number in brackets.

### Preparation of (E)-4-chloro-2-methylbut-2-en-1-ol [1]

Following the method of Hecht et al. (Hecht et al., Tetrahedron Letters, 43 (2002) 8929-8933) commercially available 2-methyl-2-vinyloxirane is converted into (E)-4-chloro-2-methylbut-2-en-1-ol [1] by treatment with TiCl4 at −80° C. to −90° C.

### Preparation of (E)-4-chloro-2-methylbut-2-en-1-(pyranyl-2′-oxy) [2]

Following the method of Miyashita et al (Miyashita et al, J. Org. Chem. 42 (1977) 3772-3774), the allylic alcohol [1] is converted into a protected form [2] by reaction of [1] with Dihydropyrane (DHP) in the presence of Pyridinium p-Toluenesulfonate (PPTs).

### Preparation of Methyl methylphosphonomorpholidate [3]

Following the method of Valentijn et al for the preparation of Farnesyl Pyrophosphate analogues (Valentijn et al, Synlett (1991) 663-664), the phosphonylating agent [3] is prepared by treatment of commercially available methylphosphonic dichloride with morpholine and methanol.

### Preparation of Intermediate [4]

Following the method of Valentijn et al (Valentijn et al, Synlett (1991) 663-664), intermediate [4] is prepared by reaction of [2] with methyl lithiomethylphosphonomorpholidate obtained in situ from the phosphonylating agent [3].

### Preparation of (E)-5-hydroxy-4-methylpent-3-enyl pyrophosphonate (C-HDMAPP)

A crude solution of C-HDMAPP is obtained in a 3 step procedure:
demethylation of intermediate [4] by treatment with tetra-n-butylammonium hydroxide in methanol as reported by Phan and Poulter (J. Org. Chem. (2001), 66, 6705-6710),
coupling with phosphoric acid following the procedure of Valentijn et al (Valentijn et al, Synlett (1991) 663-664), and
deprotection of the pyranyl-2′-oxy group by subsequent treatment of the pyrophosphonate ester with chlorhydric acid at pH 1-2 to yield a crude solution of C-HDMAPP.

The crude salt of C-HDMAPP obtained at this stage is converted to the ammonium form by cation-exchange over DOWEX 50WX8-200 resin (ammonium form). Purification of the resulting solution is performed by chromatography over silica gel using 27% ammonia solution/2-propanol 50/50 (v/v) as eluant. For the purpose of performing biological testing, the aqueous solutions of the product are sterilized by filtration through a 0.2 μm filter and stored at −20° C. In the case of testing performed in vivo, the solutions are passed beforehand through a DOWEX 50WX8-200 cationic resin column (sodium form) eluted by two column volumes of deionized water.

### Example 3

### Synthesis of BrHPP

All glassware and equipment were dried for several hours prior to use. Unless otherwise stated, the reagents and starting material were from Fluka. Trisodium (R,S)-3-(bromomethyl)-3-butanol-1-yl-diphosphate (BrHPP) was produced as white amorphous powder by the following procedure. Tosyl chloride (4.8 g, 25 mmol) and 4-(N,N-dimethylamino-) pyridine (3.4 g, 27.5 mmol; Aldrich) were mixed under magnetic stirring with 90 ml of anhydrous dichloromethane in a 250-ml three-necked flask cooled in an ice bath. A solution of 3-methyl-3-butene-1-ol (2.2 g, 25 mmol) in about 10 ml of anhydrous dichloromethane was then slowly introduced with a syringe through a septum in the flask, and the ice bath was then removed. The reaction was monitored by silica gel TLC (pentane/ethyl acetate, 85:15 (v/v)). After 2 h with constant stirring, the mixture was precipitated by dilution into 1 liter of hexane and filtered, and the filtrate was concentrated under reduced pressure. This filtration/suspension step was repeated using diethyl ether, and the resulting oil was purified by liquid chromatography on silica gel (pentane/ethyl acetate, 85:15 (v/v)), yielding a yellow oil of 3-methyl-3-butene-1-yl-tosylate (5.6 g, 23.5 mmol, 94% yield) kept under dry N₂ at 4° C. (positive mode ESI-MS: m/z 241 [M+H]⁺; m/z 258 [M+NH₄]⁺; m/z 263 [M+Na]⁺; MS² of m/z 258: m/z 190 (C₅H₈ loss)).

Disodium dihydrogen pyrophosphate (51.5 mmol, 11.1 g) dissolved in 100 ml of deionized water (adjusted to pH 9 with NH₄OH) was passed over a cation exchange DOWEX 50WX8 (42 g, 200 meq of form H⁺) column and eluted with 150 ml of deionized water (pH 9). The collected solution was neutralized to pH 7.3 using tetra-n-butyl ammonium hydroxide and lyophilized. The resulting hygroscopic powder was solubilized with anhydrous acetonitrile and further dried by repeated evaporation under reduced pressure. The resulting Tris (tetra-n-butyl ammonium) hydrogenopyrophosphate (97.5% purity by HPAEC; see below) was stored (concentration, ˜0.5 M) at −20° C. in anhydrous conditions under molecular sieves. 100 ml of a solution containing 50 mmol of Tris (tetra-n-butyl ammonium) hydrogenopyrophosphate (0.5 M, 2.5 eq) in anhydrous acetonitrile under magnetic stirring in a 250-ml three-necked flask cooled in an ice bath were slowly mixed with 20 mmol (4.8 g) of 3-methyl-3-butene-1-yl-tosylate introduced via a septum with a syringe. After 20 min, the ice bath was withdrawn, and the reaction was left under agitation at room temperature for 24 h. The reaction was analyzed by HPAEC (see below), evaporated, and diluted into 50 ml of a mixture composed of a solution (98% volume) of ammonium hydrogenocarbonate (25 mM) and 2-propanol (2 volume %). The resulting mixture was passed over a cation exchange DOWEX 50WX8 (NH₄⁺,750 meq) column formerly equilibrated with 200 ml of the solution (98% volume) of ammonium hydrogenocarbonate (25 mM) and 2-propanol (2 volume %). The column was eluted with 250 ml of the same solution at a slow flow and collected in a flask kept in an ice bath. The collected liquid was lyophilized, and the resulting white powder was solubilized in 130 ml of ammonium hydrogenocarbonate (0.1 M) and completed by 320 ml of acetonitrile/2-propanol (v/v). After agitation, the white precipitate of inorganic pyro- and mono-phosphates was eliminated by centrifugation (2100×g, 10° C., 8 min). This procedure was repeated three times, the supernatant was collected and dried, and the resulting oil was diluted in 120 ml of water. Remainders of unreacted tosylates were extracted three times by chloroform/methanol (7:3 (v/v)) in a separatory funnel, and the water phase was finally lyophilized. The resulting white powder was again washed twice by acetonitrile/chloroform/methanol (50:35:15 (v/v)) and dried under gentle N₂ flow. 11.25 mmol of pure 3-methyl-3-butene-1-yl-pyrophosphate triammonium salt were obtained by this procedure (75% yield) and were then dissolved in 200 ml of water for oxidation. For 6 mmol of 3-methyl-3-butene-1-yl-pyrophosphate, an aqueous solution of Br₂ (0.1 M) kept at 4° C. was added dropwise until appearance of a persistent yellowish color, yielding after evaporation 5.8 mmol (2.3 g) of an acidic solution (pH 2.1) of BrHPP, which was immediately neutralized by passing over DOWEX 50WX8-200 (NH₄⁺, 48 meq). The ammonium salt of BrHPP obtained after lyophilization was dissolved in water and separated from bromides by passing through Dionex OnGuard-Ag (2 meq/unit) cartridges and an on-line column of (100 meq, 21 g) DOWEX 50WX8-200 (Na⁺) eluted by milli-Q water. Colorless stock solutions of BrHPP (Na⁺) were filtered over Acrodisc 25 membranes of 0.2 μM and kept as aliquots at −20° C.

HPLC—Final purification of BrHPP was achieved by HPLC (Spectra system P1000 XR device) on an analytic Symmetry 5μ Cl8 column (Waters) eluted at 1 ml/min and 20° C. with the ternary gradient indicated below. Upstream of detectors, a split of eluent distributes 190 μl/min in the online MS detector (see below), and the remaining 810 μl/min was sent to the Waters 996 photodiode array detector. Single wavelength detection at λ=226 nm was of 7 milliabsorbance units for 6 μg of BrHPP injected in 25 μl (Rheodyne injector). The gradient program was as follows: solvent A, acetonitrile; solvent B, 50 mM ammonium acetate; solvent C, water; 0-7 min, 5% B in C, 7.1-11 min, 100% C; 12-15 min, 100% A; 15-17 min, 100% C.

### Example 4

### Administration of BrHPP for Treatment of Superficial Bladder Cancer in Humans

ImmuCyst® (Bacillus Calmette-Guérin (BCG), substrain Connaught) is commercialised by Aventis Pasteur SA, France. ImmuCyst® is made from a culture of an attenuated strain of living bovine tubercle bacillus Mycobacterium bovis. Phosphostim (Innate Pharma, Marseille, France) is based on a new chemical entity, the drug substance Bromohydrin Pyrophosphate (BrHPP), which is a specific agonist of immune competent cells namely the Vγ9Vδ2 T cell subpopulation bearing anti-tumor activity. Phosphostim (BrHPP, 200 mg) is the intravenous formulation of BrHPP for cancer immunotherapy.

Treatment begins between 7 to 14 days after biopsy or transurethral resection. On the same day, ImmuCyst® is administered as intravesical treatment of the urinary bladder and Phosphostim is administered intravenously. The induction treatment comprises 6 weekly intravesical treatments with ImmuCyst®. Each treatment dose of ImmuCyst® comprises one 81 mg vial of ImmuCyst®. A patient receives repeated cycles of Phosphostim treatment every 3 weeks. The cycle consists of one administration by infusion of Phosphostim. Phosphostim can be administered in a dose of about 200 mg/m² or about 600 mg/m², although the Phosphostim dose can be between 200 mg/m² (5 mg/kg) (corresponding to 118 mg-equivalent of BrHPP anionic form) and about 1000 mg/m² and will be determined in a dose ranging study. Optionally, each administration of Phosphostim is combined with an administration of 1 million IU/m²/day of IL-2 (for a total duration of 7 days).

ImmuCyst® is preferably dosed and administered according to the manufacturer's instructions. Each dose (1 reconstituted vial) is further diluted in an additional 50 mL of sterile, preservative-free saline for a total of 53 mL. A urethral catheter is inserted into the bladder under aseptic conditions, the bladder is drained, and then 53 mL suspension of ImmuCyst® is instilled slowly by gravity, following which the catheter is withdrawn. The patient retains the suspension for as long as possible for a total of up to two hours. During the first 15 minutes following instillation, the patient should lie prone. Thereafter, the patient is then allowed to be up. At the end of 2 hours, all patients should void in a seated position for environmental safety reasons. Patients should be instructed to maintain adequate hydration.

Phosphostim (BrHPP, 200 mg) is a freeze-dried apyrogenic sterile white powder to be reconstituted in solution for infusion. Each vial of Phosphostim (BrHPP, 200 mg) contains 200 mg of BrHPP anionic form and 50 mg the excipient alpha-lactose monohydrate (USP). Phosphostim is for immediate and single use following first opening and reconstitution. Phosphostim is reconstituted immediately prior to use with 2 ml of water for injections to make a 100 mg/ml solution. The needed quantities of reconstituted product are diluted in a total volume 100 ml of ringer lactate buffer infusion vehicle. The diluted solution is clear and colorless. Phosphostim is administered intravenously over 1 hour.

1-57. (canceled)

58. A method for treating a carcinoma or viral infection in a patient, comprising administering to a patient in need thereof an amount of a Mycobacterium antigen and a γδT cell activator effective to treat said carcinoma or viral infection.

59. The method according to claim 58, wherein said carcinoma or viral infection is superficial basal cell carcinoma, HPV infection or malignant melanoma.

60. The method according to claim 58, wherein said Mycobacterium antigen is an attenuated Mycobacterium strain.

61. The method according to claim 58, wherein said Mycobacterium antigen and said γδ T cell activator are administered simultaneously or separately and are administered by the same or different routes.

62. The method according to claim 58, wherein said Mycobacterium antigen is administered intravesicularly into the bladder.

63. The method according to claim 61, wherein said Mycobacterium antigen is administered intravesicularly into the bladder and said Mycobacterium antigen is administered simultaneously or separately with said γδ T cell activator.

64. The method according to claim 58, wherein said γδT cell activator is a compound of formula (I): 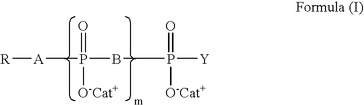
wherein Cat+ represents one (or several, identical or different) organic or mineral cation(s) (including proton);
m is an integer from 1 to 3;
B is O, NH, or any group capable to be hydrolyzed;
Y═O⁻Cat+, a C₁-C₃ alkyl group, a group -A-R, or a radical selected from the group consisting of a nucleoside, an oligonucleotide, a nucleic acid, an amino acid, a peptide, a protein, a monosaccharide, an oligosaccharide, a polysaccharide, a fatty acid, a simple lipid, a complex lipid, a folic acid, a tetrahydrofolic acid, a phosphoric acid, an inositol, a vitamin, a co-enzyme, a flavonoid, an aldehyde, an epoxyde and a halohydrin;
A is O, NH, CHF, CF₂ or CH₂; and,
R is a linear, branched, or cyclic, aromatic or not, saturated or unsaturated, C₁-C₅₀ hydrocarbon group, optionally interrupted by at least one heteroatom, wherein said hydrocarbon group comprises an alkyl, an alkylenyl, or an alkynyl, preferably an alkyl or an alkylene, which can be substituted by one or several substituents selected from the group consisting of: an alkyl, an alkylenyl, an alkynyl, an epoxyalkyl, an aryl, an heterocycle, an alkoxy, an acyl, an alcohol, a carboxylic group (—COOH), an ester, an amine, an amino group (—NH₂), an amide (—CONH₂), an imine, a nitrile, an hydroxyl (—OH), a aldehyde group (—CHO), an halogen, an halogenoalkyl, a thiol (—SH), a thioalkyl, a sulfone, a sulfoxide, and a combination thereof.

65. The method according to claim 63, wherein said γδT cell activator is a compound of formula (I): 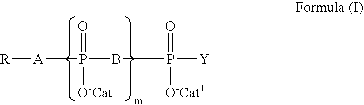
wherein Cat+ represents one (or several, identical or different) organic or mineral cation(s) (including proton);
m is an integer from 1 to 3;
B is O, NH, or any group capable to be hydrolyzed;
Y═O⁻Cat+, a C₁-C₃ alkyl group, a group -A-R, or a radical selected from the group consisting of a nucleoside, an oligonucleotide, a nucleic acid, an amino acid, a peptide, a protein, a monosaccharide, an oligosaccharide, a polysaccharide, a fatty acid, a simple lipid, a complex lipid, a folic acid, a tetrahydrofolic acid, a phosphoric acid, an inositol, a vitamin, a co-enzyme, a flavonoid, an aldehyde, an epoxyde and a halohydrin;
A is O, NH, CHF, CF₂ or CH₂; and,
R is a linear, branched, or cyclic, aromatic or not, saturated or unsaturated, C₁-C₅₀ hydrocarbon group, optionally interrupted by at least one heteroatom, wherein said hydrocarbon group comprises an alkyl, an alkylenyl, or an alkynyl, preferably an alkyl or an alkylene, which can be substituted by one or several substituents selected from the group consisting of: an alkyl, an alkylenyl, an alkynyl, an epoxyalkyl, an aryl, an heterocycle, an alkoxy, an acyl, an alcohol, a carboxylic group (—COOH), an ester, an amine, an amino group (—NH₂), an amide (—CONH₂), an imine, a nitrile, an hydroxyl (—OH), a aldehyde group (—CHO), an halogen, an halogenoalkyl, a thiol (—SH), a thioalkyl, a sulfone, a sulfoxide, and a combination thereof.

66. The method according to claim 58, where said γδT cell activator is a compound of formula (II): 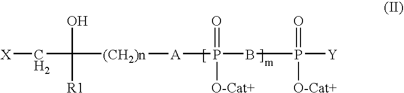
in which X is an halogen (preferably selected from I, Br and Cl), B is O or NH, m is an integer from 1 to 3, R1 is a methyl or ethyl group, Cat+ represents one (or several, identical or different) organic or mineral cation(s) (including the proton), and n is an integer from 2 to 20, A is O, NH, CHF, CF₂ or CH₂, and Y is O⁻Cat+, or a nucleoside.

67. The method according to claim 66, wherein the compound of formula (II) is selected from the group consisting of BrHPP, CBrHPP and epoxPP.

68. The method according to claim 63, wherein said γδT cell activator is a compound of formula (II): 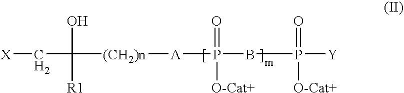
in which X is an halogen (preferably selected from I, Br and Cl), B is O or NH, m is an integer from 1 to 3, R1 is a methyl or ethyl group, Cat+ represents one (or several, identical or different) organic or mineral cation(s) (including the proton), and n is an integer from 2 to 20, A is O, NH, CHF, CF₂ or CH₂, and Y is O⁻Cat+, or a nucleoside.

69. The method according to claim 68, wherein the compound of formula (II) is selected from the group consisting of BrHPP, CBrHPP and epoxPP.

70. The method according to claim 58, wherein said γδT cell activator is a compound of formula (XII): 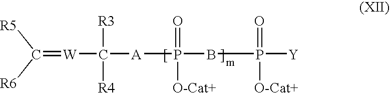
in which R₃, R₄, and R₅, identical or different, are a hydrogen or (C₁-C₃)alkyl group, W is —CH— or —N—, R₆ is an (C₂-C₃)acyl, an aldehyde, an (C₁-C₃)alcohol, or an (C₂-C₃)ester, Cat+ represents one (or several, identical or different) organic or mineral cation(s) (including the proton), B is O or NH, m is an integer from 1 to 3, A is O, NH, CHF, CF₂ or CH₂, and Y is O⁻Cat+, or a nucleoside.

71. The method according to claim 70, wherein the compound of formula (XII) is HDMAPP or CHDMAPP.

72. The method according to claim 63, wherein said γδT cell activator is a compound of formula (XII): 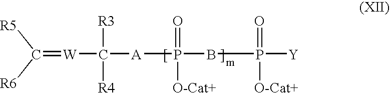
in which R₃, R₄, and R₅, identical or different, are a hydrogen or (C₁-C₃)alkyl group, W is —CH— or —N—, R₆ is an (C₂-C₃)acyl, an aldehyde, an (C₁-C₃)alcohol, or an (C₂-C₃)ester, Cat+ represents one (or several, identical or different) organic or mineral cation(s) (including the proton), B is O or NH, m is an integer from 1 to 3, A is O, NH, CHF, CF₂ or CH₂, and Y is O⁻Cat+, or a nucleoside.

73. The method according to claim 72, wherein the compound of formula (XII) is HDMAPP or CHDMAPP.

74. A method for treating a disease comprising in a subject, comprising:
(a) administering to said subject a γδ T cell activator compound; and
(b) administering to a subject locally at a site of disease an immunomodulatory composition (IMC) or immunogenic composition (IC).

75. The method according to claim 74, wherein the IC or IMC is a Mycobacterium antigen.

76. The method according to claim 75, wherein said Mycobacterium antigen is an attenuated Mycobacterium strain.

77. The method according to claim 75, wherein said Mycobacterium antigen and said γδ T cell activator are administered simultaneously or separately and are administered by the same or different routes.

78. The method according to claim 75, wherein said Mycobacterium antigen is administered intravesicularly into the bladder.

79. The method according to claim 75, wherein said Mycobacterium antigen is administered intravesicularly into the bladder and said Mycobacterium antigen is administered simultaneously or separately with said γδ T cell activator.

80. The method according to claim 74, wherein said γδT cell activator is a compound of formula (I): 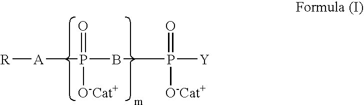
wherein Cat+ represents one (or several, identical or different) organic or mineral cation(s) (including proton);
m is an integer from 1 to 3;
B is O, NH, or any group capable to be hydrolyzed;
Y═O⁻Cat+, a C₁-C₃ alkyl group, a group -A-R, or a radical selected from the group consisting of a nucleoside, an oligonucleotide, a nucleic acid, an amino acid, a peptide, a protein, a monosaccharide, an oligosaccharide, a polysaccharide, a fatty acid, a simple lipid, a complex lipid, a folic acid, a tetrahydrofolic acid, a phosphoric acid, an inositol, a vitamin, a co-enzyme, a flavonoid, an aldehyde, an epoxyde and a halohydrin;
A is O, NH, CHF, CF₂ or CH₂; and,
R is a linear, branched, or cyclic, aromatic or not, saturated or unsaturated, C₁-C₅₀ hydrocarbon group, optionally interrupted by at least one heteroatom, wherein said hydrocarbon group comprises an alkyl, an alkylenyl, or an alkynyl, preferably an alkyl or an alkylene, which can be substituted by one or several substituents selected from the group consisting of: an alkyl, an alkylenyl, an alkynyl, an epoxyalkyl, an aryl, an heterocycle, an alkoxy, an acyl, an alcohol, a carboxylic group (—COOH), an ester, an amine, an amino group (—NH₂), an amide (—CONH₂), an imine, a nitrile, an hydroxyl (—OH), a aldehyde group (—CHO), an halogen, an halogenoalkyl, a thiol (—SH), a thioalkyl, a sulfone, a sulfoxide, and a combination thereof.

81. The method according to claim 74, wherein said γδT cell activator is a compound of formula (II): 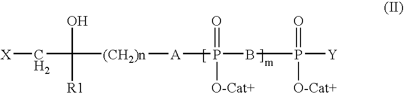
in which X is an halogen (preferably selected from I, Br and Cl), B is O or NH, m is an integer from 1 to 3, R1 is a methyl or ethyl group, Cat+ represents one (or several, identical or different) organic or mineral cation(s) (including the proton), and n is an integer from 2 to 20, A is O, NH, CHF, CF₂ or CH₂, and Y is O⁻Cat+, or a nucleoside.

82. The method according to claim 81, wherein the compound of formula (II) is selected from the group consisting of BrHPP, CBrHPP and epoxPP.

83. The method according to claim 74, where said γδT cell activator is a compound of formula (XII): 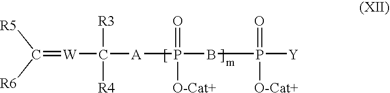
in which R₃, R₄, and R₅, identical or different, are a hydrogen or (C₁-C₃)alkyl group, W is —CH— or —N—, R₆ is an (C₂-C₃)acyl, an aldehyde, an (C₁-C₃)alcohol, or an (C₂-C₃)ester, Cat+ represents one (or several, identical or different) organic or mineral cation(s) (including the proton), B is O or NH, m is an integer from 1 to 3, A is O, NH, CHF, CF₂ or CH₂, and Y is O⁻Cat+, or a nucleoside.

84. The method according to claim 83, wherein the compound of formula (XII) is HDMAPP or CHDMAPP.

85. The method according to claim 74, wherein:
a) the IMC comprises:
i) a compound which is an agonist of a toll-like receptor (TLR);
ii) a compound which is an agonist of a toll-like receptor (TLR) selected from the group consisting of TLR2, TLR3, TLR4, TLR6, TLR7, TLR8, TLR9 and TLR10;
iii) a cytokine;
iv) a cytokine is selected from the group consisting of IL-2, IL-4, IL-5, IL-6, IL-7, IL-8, IL-9, IL-10, IL-11, IL-12, IL-13, IL-15, IL-18 and IL-21;
v) a compound which is an imidazoquinoline compound or analog or derivative thereof; or
vi) a CpG nucleic acid, or analog or derivative thereof; or
b) the IC comprises a cancer antigen or a bacterial antigen.

86. The method according to claim 74, wherein said medicament is for the treatment of superficial basal cell carcinoma, HPV infection or malignant melanoma.

87. The method according to claim 80, wherein:
a) the IMC comprises:
i) a compound which is an agonist of a toll-like receptor (TLR);
ii) a compound which is an agonist of a toll-like receptor (TLR) selected from the group consisting of TLR2, TLR3, TLR4, TLR6, TLR7, TLR8, TLR9 and TLR10;
iii) a cytokine;
iv) a cytokine is selected from the group consisting of IL-2, IL-4, IL-5, IL-6, IL-7, IL-8, IL-9, IL-10, IL-11, IL-12, IL-13, IL-15, IL-18 and IL-21;
v) a compound which is an imidazoquinoline compound or analog or derivative thereof; or
vi) a CpG nucleic acid, or analog or derivative thereof; or
b) the IC comprises a cancer antigen or a bacterial antigen.

88. The method according to claim 81, wherein:
a) the IMC comprises:
i) a compound which is an agonist of a toll-like receptor (TLR);
ii) a compound which is an agonist of a toll-like receptor (TLR) selected from the group consisting of TLR2, TLR3, TLR4, TLR6, TLR7, TLR8, TLR9 and TLR10;
iii) a cytokine;
iv) a cytokine is selected from the group consisting of IL-2, IL-4, IL-5, IL-6, IL-7, IL-8, IL-9, IL-10, IL-11, IL-12, IL-13, IL-15, IL-18 and IL-21;
v) a compound which is an imidazoquinoline compound or analog or derivative thereof; or
vi) a CpG nucleic acid, or analog or derivative thereof; or
b) the IC comprises a cancer antigen or a bacterial antigen.

89. The method according to claim 83, wherein:
a) the IMC comprises:
i) a compound which is an agonist of a toll-like receptor (TLR);
ii) a compound which is an agonist of a toll-like receptor (TLR) selected from the group consisting of TLR2, TLR3, TLR4, TLR6, TLR7, TLR8, TLR9 and TLR10;
iii) a cytokine;
iv) a cytokine is selected from the group consisting of IL-2, IL-4, IL-5, IL-6, IL-7, IL-8, IL-9, IL-10, IL-11, IL-12, IL-13, IL-15, IL-18 and IL-21;
v) a compound which is an imidazoquinoline compound or analog or derivative thereof; or
vi) a CpG nucleic acid, or analog or derivative thereof; or
b) the IC comprises a cancer antigen or a bacterial antigen.

90. An article of manufacture comprising:
a) a pharmaceutical composition comprising a Mycobacterium antigen and a γδ T cell activator at an effective dose to treat a carcinoma or viral infection;
b) a kit comprising a pharmaceutical composition comprising an IC or IMC and a pharmaceutical composition comprising a γδ T cell activator, said compositions at effective doses to treat a carcinoma or viral infection when used together in combination therapy and wherein the IC or IMC is provided in a form suitable for local administration to a site of disease; or
c) a kit comprising a pharmaceutical composition comprising a Mycobacterium antigen and a pharmaceutical composition comprising a γδ T cell activator, said compositions at effective doses to treat a carcinoma or viral infection when used together in combination therapy and wherein the IC or IMC is provided in a form suitable for local administration to a site of disease.

91. The article of manufacture according to claim 90, wherein said Mycobacterium antigen is an attenuated Mycobacterium strain.

92. The article of manufacture according to claim 90, wherein said γδT cell activator is a compound of formula (I): 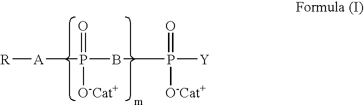
wherein Cat+ represents one (or several, identical or different) organic or mineral cation(s) (including proton);
m is an integer from 1 to 3;
B is O, NH, or any group capable to be hydrolyzed;
Y═O⁻Cat+, a C₁-C₃ alkyl group, a group -A-R, or a radical selected from the group consisting of a nucleoside, an oligonucleotide, a nucleic acid, an amino acid, a peptide, a protein, a monosaccharide, an oligosaccharide, a polysaccharide, a fatty acid, a simple lipid, a complex lipid, a folic acid, a tetrahydrofolic acid, a phosphoric acid, an inositol, a vitamin, a co-enzyme, a flavonoid, an aldehyde, an epoxyde and a halohydrin;
A is O, NH, CHF, CF₂ or CH₂; and,
R is a linear, branched, or cyclic, aromatic or not, saturated or unsaturated, C₁-C₅₀ hydrocarbon group, optionally interrupted by at least one heteroatom, wherein said hydrocarbon group comprises an alkyl, an alkylenyl, or an alkynyl, preferably an alkyl or an alkylene, which can be substituted by one or several substituents selected from the group consisting of: an alkyl, an alkylenyl, an alkynyl, an epoxyalkyl, an aryl, an heterocycle, an alkoxy, an acyl, an alcohol, a carboxylic group (—COOH), an ester, an amine, an amino group (—NH₂), an amide (—CONH₂), an imine, a nitrile, an hydroxyl (—OH), a aldehyde group (—CHO), an halogen, an halogenoalkyl, a thiol (—SH), a thioalkyl, a sulfone, a sulfoxide, and a combination thereof.

93. The article of manufacture according to claim 90, where said γδT cell activator is a compound of formula (II): 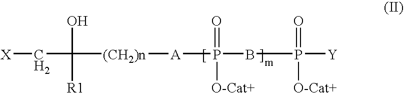
in which X is an halogen (preferably selected from I, Br and Cl), B is O or NH, m is an integer from 1 to 3, R1 is a methyl or ethyl group, Cat+ represents one (or several, identical or different) organic or mineral cation(s) (including the proton), and n is an integer from 2 to 20, A is O, NH, CHF, CF₂ or CH₂, and Y is O⁻Cat+, or a nucleoside.

94. The article of manufacture according to claim 93, wherein the compound of formula (II) is selected from the group consisting of BrHPP, CBrHPP and epoxPP.

95. The article of manufacture according to claim 90, where said γδT cell activator is a compound of formula (XII): 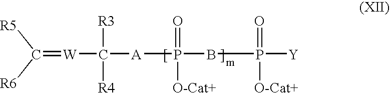
in which R₃, R₄, and R₅, identical or different, are a hydrogen or (C₁-C₃)alkyl group, W is —CH— or —N—, R₆ is an (C₂-C₃)acyl, an aldehyde, an (C₁-C₃)alcohol, or an (C₂-C₃)ester, Cat+ represents one (or several, identical or different) organic or mineral cation(s) (including the proton), B is O or NH, m is an integer from 1 to 3, A is O, NH, CHF, CF₂ or CH₂, and Y is O⁻Cat+, or a nucleoside.

96. The article of manufacture according to claim 95, wherein the compound of formula (XII) is HDMAPP or CHDMAPP.
